(19) United States
(12) Patent Application Publication
(10) Pub. No.: US 2022/0177905 A1
(43) Pub. Date: Jun. 9, 2022

(54) Compositions and Methods for Allelic Gene Drive Systems and Lethal Mosaicism

(71) Applicant: The Regents of the University of California, Oakland, CA (US)

(72) Inventors: Ethan Bier, San Diego, CA (US); Annabel Guichard, La Jolla, CA (US); Valentino Gantz, San Diego, CA (US)

(21) Appl. No.: 17/600,820

(22) PCT Filed: Apr. 3, 2020
(86) PCT No.: PCT/US2020/026522

§ 371 Date: Oct. 1, 2021

Related U.S. Application Data
(60) Provisional application No. 62/828,544, filed on Apr. 3, 2019.

(51) Int. Cl. C12N 15/82; C12N 15/10; C12N 15/90

(52) U.S. Cl. CPC C12N 15/8279; C12N 15/90; C12N 15/102

(57) ABSTRACT

An active genetic approach to preferentially transmit allelic variants (allelic-drive) resulting from only a single or a few nucleotide alterations. Embodiments are provided for allelic-drive: one, copy-cutting, in which a non-preferred allele is selectively targeted for Cas9/guide RNA (gRNA) cleavage, and a more general approach, copy-grafting, that permits selective inheritance of a desired allele located at some distance from the gRNA cut site. A lethal mosaicism is provided that dominantly eliminates NHEJ-induced mutations and favors inheritance of functional cleavage-resistant alleles. These two efficient allelic-drive methods, enhanced by lethal mosaicism and a trans-generational drive process provide a shadow-drive, that are applicable to improvements in health and agriculture.

### CROSS REFERENCES TO RELATED APPLICATIONS

This application claims the priority benefit of U.S. Provisional Application No. 62/828,544, filed Apr. 3, 2019, which application is incorporated herein by reference.

### GOVERNMENT SPONSORSHIP

This invention was made with government support under grant No. GM11732I awarded by the National Institutes of Health. The government has certain rights in the invention.

### TECHNICAL FIELD

The present invention relates to compositions and methods for allelic gene drive systems and lethal mosaicism.

### BACKGROUND

Efficient super-Mendelian inheritance of transgenic insertional elements has been demonstrated in flies, mosquitoes, yeast, and mice¹⁻⁵. While numerous potentially impactful applications of such so-called gene-drive systems have been proposed^{6,7}, they are currently limited to copying relatively large DNA cargo sequences (˜1-10 Kb). Many desired genetic traits (e.g., drought tolerance in plants, crop yield, pest-resistance, or insecticide sensitivity), however, result from allelic variants altering only one or a few base pairs. An efficient system for super-Mendelian inheritance of such subtle genetic variants would accelerate a wide array of efforts to disseminate favorable traits throughout populations, or to assemble complex genotypes consisting of point-mutant alleles in combination with insertional transgenes for a multitude of research and applied purposes.

Widely used CRISPR-Cas9-based gene editing approaches⁸ involve enzymatic cleavage of a sensitive allele and repair by copying information from an exogenously provided cut-resistant oligonucleotide or double-stranded DNA template^{9,10}.

### SUMMARY OF THE INVENTION

The invention provides methods and compositions for germline editing in heterozygous individuals carrying two different alleles of a gene. In embodiments, the invention provides methods and compositions to repair a cleavage sensitive allele with sequences provided by a cut-resistant allele present on the companion chromosome. This type of allelic-drive can supplement a gene-drive system that copies itself in one genomic location, by adding a second guide RNA (gRNA) to the gene-drive cassette that directs selective cleavage of a non-preferred allele at a separate genomic site. Such a dual gRNA drive system (e.g., FIGS. 1A-1B) results in efficient super-Mendelian inheritance of both to the gene-drive element and the beneficial allelic variant via germline transmission when carrier individuals mate with recipient individuals bearing the undesired cleavage-sensitive allele.

The invention provides at least two forms of allelic drive. The first, copy-cutting involves a Cas9-gRNA complex selectively cutting one allelic variant, followed by HDR-mediated repair and replacement with a non-cleavable allele of the same gene provided in-trans. The second and more generally applicable form of allelic-drive, copy-grafting, involves copying a short genomic interval that encompasses a favored allele in proximity to a gRNA cut site. In the case of copy-grafting, the favored allele is associated with neighboring sequences resistant to gRNA cleavage.

The invention further provides a technique referred to as lethal-mosaicism, that dominantly eliminates NHEJ-induced mutations and favors inheritance of functional cleavage-resistant alleles. The basis for lethal mosaicism is that mutant alleles produced by non-homologous end-joining (NHEJ) in an essential gene become dominantly lethal during the drive process. In contrast, a protected non-cleavable functional allele of the gene, remains immune to such lethal mosaicism. Thus, lethal mosaicism results in selective elimination of undesired alleles generated by NHEJ.

The disclosure provides a method of introducing a desired nucleotide sequence into a genome by copy-cutting, comprising genomically integrating the desired nucleotide sequence into a selected allele by targeting an undesired nucleic acid sequence for Cas/guide RNA cleavage; and replacing therewith a non-cleavable portion of the corresponding allele containing the desired nucleotide sequence.

In embodiments, the invention provides a method of introducing a desired nucleotide sequence into a genome by copy-grafting, comprising genomically integrating the desired sequence into a selected allele by targeting an undesired nucleic acid sequence at a distance from Cas/guide RNA cleavage; and replacing therewith a non-cleavable portion of the corresponding allele containing the desired nucleotide sequence and a genomic interval.

In embodiments, the invention provides a method of selectively eliminating to an allele of a gene generated by non-homologous end joining (NHEJ) comprising utilizing a lethal mosaicism technique described herein to create a protected, non-cleavable functional allele.

In embodiments, the invention provides a method of producing a protected non-cleavable functional allele of a gene comprising utilizing a lethal mosaicism technique described herein.

### BRIEF DESCRIPTION OF THE DRAWINGS

FIGS. 1A-1F show Super-Mendelian inheritance of a dominant Notch allele in Drosophila.

FIGS. 2A-2E show drive-induced lethal events on the receiver chromosome.

FIGS. 3A-3D show shadow drive and co-drive analyses.

FIGS. 4A-4D show an allelic drive mediated by “copy-grafting”.

FIG. 5 shows a circular map of ccN CopyCat element plasmid and a linear map showing integration of the ccN element into the yellow locus and the locations of primers used to PCR amplify fragments for sequencing.

FIG. 6 shows a sequence analysis of Cas9-induced yellow-mutations on receiver chromosomes.

FIG. 7 shows a mosaic phenotypes of F2 female progeny of master females.

FIGS. 8A-8B show molecular and genetic makers for scoring donor versus receiver chromosomes.

FIGS. 9A-9C show sequence analysis of Cas9-induced Notch mutations and allelic variants.

FIG. 10 shows sequences of genomic DNA mutations and resulting amino acid alterations in various NHEJ-induced Notch alleles.

FIG. 11 shows rescuing lethal mosaicism in female F2 progeny skews the fraction of receiver chromosomes recovered in males.

FIGS. 12A-12B show 100% lethality induced by the ccN CopyCat element in presence of two sensitive Notch wild-type alleles and Cas9.

FIGS. 13A-13B show allelic conversion at the Notch locus is reduced when copying of the ccN element at yellow is blocked.

FIGS. 14A-14B show efficient allelic drive is observed when the N^{Ax16} allele is in a trans-configuration relative to the y^{<ccN>} allele.

FIG. 15 shows efficient allelic drive of the de novo cleavage site N^{Ax103} allele.

FIG. 16 shows copy-grafting sustains efficient co-drive.

FIG. 17 shows the nanos-Cas9 source sustains efficient allelic conversion, but also induces more male lethal NHEJ events than observed with the vasa-Cas9 source.

FIGS. 18A-18B show preliminary cage trial experiments for copy-grafting allelic-drive in a Cas9 recipient population.

FIGS. 19A-19 show preliminary cage trial experiments for copy grafting in an allelic pump paradigm where a fixed percentage of Cas9+drive cassette (introduced at equal frequencies) are released into a naïve (non Cas9) recipient population.

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012). The disclosure incorporates by reference the entirety of WO2016/073559.

In embodiments, the invention provides methods of introducing a preferred nucleotide sequence into a genome by allelic-drive copy-cutting. In embodiments, allelic-drive copy-cutting comprises genomically replacing a non-preferred nucleotide sequence with that of a preferred allele of the same gene using an allelic-drive element that comprises first and second guide RNAs and a cleavage resistant preferred allele, wherein a Cas endonuclease guided by the first guide RNA copies the allelic-drive element, and a Cas endonuclease guided by the second guide RNA cuts the non-preferred allele, but not the preferred allele, and copies the cleavage resistant preferred allele into a double stranded DNA break created by the second guide RNA by homology-directed repair (HDR)-mediated repair.

In embodiments, the invention provides a method of propagating a preferred allele in a genome by allelic-drive copy-cutting, comprising: a) integrating into a first chromosome, containing a preferred allele, an allelic-drive element comprising a first guide RNA, a second guide RNA, and a cleavage insensitive portion of the preferred allele, wherein the first guide RNA cleaves the first chromosome and inserts the allelic-drive element into the first chromosome with a Cas endonuclease; and b) integrating into a second chromosome, containing a non-preferred allele of the preferred allele, the allelic-drive element, wherein the first guide RNA cleaves the second chromosome and inserts the allelic-drive element into the second chromosome with a Cas endonuclease, and the second guide RNA cleaves the second chromosome at a sensitive portion of the non-preferred allele, but not the insensitive portion of the preferred allele, with a Cas endonuclease and by homology-directed repair (HDR)-mediated repair.

In embodiments, the allelic-drive can be inserted into any locus, whether on the same chromosome as the preferred allele or not. In embodiments, the allelic-drive element can be on the first chromosome and the preferred allele can be on either the first or second chromosome. In embodiments, for example in Drosophila, the first chromosome can be X and the second chromosome can be an autosome, there also being third and fourth autosomal chromosomes. In embodiments, the allelic-drive element is inserted into the genome in a genetic background that carries the cleavage resistant or insensitive version of the preferred allele, to avoid the gRNA cutting the target gene and creating a mutation by NHEJ.

In embodiments, allelic-drive copy-cutting provides allelic conversion of both alleles in a second filial generation is at least 40%, 50%, 60% or 70%.

In embodiments, the invention provides methods of introducing a preferred nucleotide sequence into a genome by allelic-drive copy-grafting. In embodiments, the allelic-drive copy-grafting comprises genomically replacing a non-preferred nucleotide sequence with that of a preferred allele of the same gene using an allelic-drive element that comprises a first and a second guide RNAs and a cleavage resistant site adjacent to the preferred allele, wherein a Cas endonuclease guided by the first guide RNA copies the allelic-drive element, and a Cas endonuclease guided by the second guide RNA cuts the non-preferred allele, but not the preferred allele, that is associated with the cleavage resistant site residing within less than 100 nucleotides of the preferred allele, and copies the cleavage resistant sequence together with the preferred adjacent allele as an intact “island” by virtue of the short range single stranded resection step which occurs during homology-directed repair (HDR)-mediated repair.

In embodiments, the invention provides methods of introducing a preferred nucleotide sequence into a genome by allelic-drive copy-grafting. In embodiments, the allelic-drive copy-grafting comprising: a) integrating into a first chromosome, containing a preferred allele, an allelic-drive element comprising a first guide RNA, a second guide RNA, and a cleavage insensitive portion of the preferred allele, wherein the first guide RNA cleaves the first chromosome and inserts the allelic-drive element into the first chromosome with a Cas endonuclease; and b) integrating into a second chromosome, containing a non-preferred allele of the preferred allele, the allelic-drive element, wherein the first guide RNA cleaves the second chromosome and inserts the allelic-drive element into the second chromosome with a Cas endonuclease, and the second guide RNA cleaves the second chromosome at a sensitive portion within less than 100 nucleotides of the non-preferred allele with a Cas endonuclease and by homology-directed repair (HDR)-mediated repair.

In embodiments, the allelic-drive can be inserted into any locus, whether on the same chromosome as the preferred allele or not. In embodiments, the allelic-drive element can be on the first chromosome and the preferred allele can be on either the first or second chromosome. In embodiments, in the allelic-drive copy-grafting methods, a cleavage insensitive site can be created adjacent to the preferred allele, and Cas9 induced DNA breaks directed by guide RNA-mediated cleavage of chromosomes carrying either the non-preferred allele or a cleavage-sensitive preferred allele (perhaps a wild-type allele) are repaired by copying an intact region of 5 to 100 nucleotides (or base pairs) that includes the preferred allele. This embodiment replaces both non-preferred and cleavage sensitive preferred alleles with the cleavage resistance preferred allele. In embodiments, the second guide RNA cleaves the second chromosome at a cleavage sensitive portion within less than 80, 60, 40, 25, 20, 10 or 5 nucleotides from the non-preferred allele.

In embodiments, allelic-drive copy-grafting provides allelic conversion of both alleles in a second filial generation is at least 80%, 85%, 90%, 95% or 97%.

In embodiments of the allelic-drive copy-cutting and copy-grafting methods of the invention, the Cas endonuclease is not integrated into the allelic-drive element. In embodiments, the Cas endonuclease is integrated into the allelic drive element and transmission of both alleles is super-Mendelian.

In embodiments of the allelic-drive copy-cutting and copy-grafting methods of the invention, the progeny, which maintain an association between a first allele comprising an allelic-drive element and a second uncleavable (or cleavage insensitive or cleavage resistant) allele, survive in the presence of Cas9.

In embodiments of the allelic-drive copy-cutting and copy-grafting methods of the invention, perduring Cas9-gRNA complexes are transmitted maternally for one generation in the absence of Cas9 or gRNA transgenes.

In embodiments, the prodigy result from a lethal mosaicism. In embodiments of the allelic-drive copy-cutting and copy-grafting methods of the invention, the allelic-drive element is inserted into an essential gene required for viability or fertility and also carries functional recoded sequences of the essential gene rendering the allelic drive element viable in a homozygous or hemizygous state. In embodiments of the allelic-drive copy-cutting and copy-grafting methods of the invention, the functional recoded sequences of the essential gene are recoded as a direct in-frame fusion with recoded cDNA sequences inserted at the 5 end of the allelic drive element abutting the guide RNA cut site. In embodiments, the essential gene is for example Notch, Rab11, Rab5, Rab1; Prosalpha1, Dpp=decapentaplegic, EGF-Receptor; Mre11, Spo11, cinnabar=kynurenine hydroxylase; doublesex; a gene encoding a male-specific tubulin subunit; cardinal, ENa=sodium ion channel, or FREP1.

In embodiments the invention provides methods of selectively eliminating an allele of a gene generated by non-homologous end joining (NHEJ) comprising utilizing a lethal mosaicism technique, wherein NHEJ-induced drive-resistant, non-functional, or loss-of-function alleles of an essential gene are dominantly eliminated in progeny due to maternal perdurance of Cas/guide RNA complexes targeting the paternal allele.

In embodiments the invention provides methods of germline editing comprising repair of a cleavage-sensitive allele with sequences provided by a cut-resistant allele present on the homologous chromosome in heterozygous individuals.

In embodiments of the invention, the alleles are assembled in plants to provide a trait of interest, such as but not limited to, drought resistance, insect resistance, salinity resistance, higher crop yields, optimal architectures, or rapid growth.

In embodiments of the invention, the alleles are assembled in insects to reverse pesticide resistance in pest species or to favor genetic variants that prevent host species from serving as disease vectors.

In embodiments, the invention provides organism made by a method of any of the inventions and methods described herein. In embodiments, the organism is eukaryotic, such as a plant, an insect, an agricultural pest, or an animal, including a human.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable earner” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

“Complementaty” or “complement thereof” means that a contiguous nucleic acid base sequence is capable of hybridizing to another base sequence by standard base pairing (hydrogen bonding) between a series of complementary bases. Complementary sequences may be completely complementary (i.e. no mismatches in the nucleic acid duplex) at each position in an oligomer sequence relative to its target sequence by using standard base pairing (e.g., G:C, A:T or A:U pairing) or sequences may contain one or more positions that are not complementary by base pairing (e.g., there exists at least one mismatch or unmatched base in the nucleic acid duplex), but such sequences are sufficiently complementary because the entire oligomer sequence is capable of specifically hybridizing with its target sequence in appropriate hybridization conditions (i.e. partially complementary). Contiguous bases in an oligomer are typically at least 80%, preferably at least 90%, and more preferably completely complementary to the intended target sequence.

“Configured to” or “designed to” denotes an actual arrangement of a nucleic acid sequence configuration of a referenced oligonucleotide. For example, a primer that is configured to generate a specified amplicon from a target nucleic acid has a nucleic acid sequence that hybridizes to the target nucleic acid or a region thereof and can be used in an amplification reaction to generate the amplicon. Also as an example, an oligonucleotide that is configured to specifically hybridize to a target nucleic acid or a region thereof has a nucleic acid sequence that specifically hybridizes to the referenced sequence under stringent hybridization conditions.

“Region” refers to a portion of a nucleic acid wherein said portion is smaller than the entire nucleic acid.

“Region of interest” refers to a specific sequence of a target nucleic acid that includes all codon positions having at least one single nucleotide substitution mutation associated with a genotype and/or subtype that are to be modified, and all marker positions that are to be amplified and detected, if any.

“RNA-dependent DNA polymerase” or “reverse transcriptase” (“RT”) refers to an enzyme that synthesizes a complementary DNA copy from an RNA template. All known reverse transcriptases also have the ability to make a complementary DNA copy from a DNA template; thus, they are both RNA- and DNA-dependent DNA polymerases. RTs may also have an RNAse H activity. A primer is required to initiate synthesis with both RNA and DNA templates.

“DNA-dependent DNA polymerase” is an enzyme that synthesizes a complementary DNA copy from a DNA template. Examples are DNA polymerase I from E. coli, bacteriophage T7 DNA polymerase, or DNA polymerases from bacteriophages T4, Phi-29, M2, or T5. DNA-dependent DNA polymerases may be the naturally occurring enzymes isolated from bacteria or bacteriophages or expressed recombinantly, or may be modified or “evolved” forms which have been engineered to possess certain desirable characteristics, e.g., thermostability, or the ability to recognize or synthesize a DNA strand from various modified templates. All known DNA-dependent DNA polymerases require a complementary primer to initiate synthesis. It is known that under suitable conditions a DNA-dependent DNA polymerase may synthesize a complementary DNA copy from an RNA template. RNA-dependent DNA polymerases typically also have DNA-dependent DNA polymerase activity.

“DNA-dependent RNA polymerase” or “transcriptase” is an enzyme that synthesizes multiple RNA copies from a double-stranded or partially double-stranded DNA molecule having a promoter sequence that is usually double-stranded. The RNA molecules (“transcripts”) are synthesized in the 5′-to-3′ direction beginning at a specific position just downstream of the promoter. Examples of transcriptases are the DNA-dependent RNA polymerase from E. coli and bacteriophages T7, T3, and SP6.

A “sequence” of a nucleic acid refers to the order and identity of nucleotides in the nucleic acid. A sequence is typically read in the 5′ to 3′ direction. The terms “identical” or percent “identity” in the context of two or more nucleic acid or polypeptide sequences, refer to two or more sequences or subsequences that are the same or have a specified percentage of amino acid residues or nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al, (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated by reference. Many other optimal alignment algorithms are also known in the art and are optionally utilized to determine percent sequence identity.

A “label” refers to a moiety attached (covaleinly or non-covidently), or capable of being attached, to a molecule, which moiety provides or is capable of providing information about the molecule (e.g., descriptive, identifying, etc. information about the molecule) or another molecule with which the labeled molecule interacts (e.g., hybridizes, etc.). Exemplary labels include fluorescent labels (including, e.g., quenchers or absorbers), weakly fluorescent labels, non-fluorescent labels, colorimetric labels, chemiluminescent labels, bioluminescent labels, radioactive labels, mass-modifying groups, antibodies, antigens, biotin, haptens, enzymes (including, e.g., peroxidase, phosphatase, etc.), and the like.

A “linker” refers to a chemical moiety that covalently or non-coyalently attaches a compound or substituent group to another moiety, e.g., a nucleic acid, an oligonucleotide probe, a primer nucleic acid, an amplicon, a solid support, or the like. For example, linkers are optionally used to attach oligonucleotide probes to a solid support (e.g., in a linear or other logic probe array). To further illustrate, a linker optionally attaches a label (e.g., a fluorescent dye, a radioisotope, etc.) to an oligonucleotide probe, a primer nucleic acid, or the like. Linkers are typically at least bifunctional chemical moieties and in certain embodiments, they comprise cleavable attachments, which can be cleaved by, e.g., heat, an enzyme, a chemical agent, electromagnetic radiation, etc. to release materials or compounds from, e.g., a solid support. A careful choice of linker allows cleavage to be performed under appropriate conditions compatible with the stability of the compound and assay method. Generally a linker has no specific biological activity other than to, e.g., join chemical species together or to preserve some minimum distance or other spatial relationship between such species. However, the constituents of a linker may be selected to influence some property of the linked chemical species such as three-dimensional conformation, net charge, hydrophobicity, etc. Exemplary linkers include, e.g., oligopeptides, oligonucleotides, oligopolyamides, oligoethyleneglycerols, oligoacrylamides, alkyl chains, or the like. Additional description of linker molecules is provided in, e.g., Hermanson, Bioconjugate Techniques, Elsevier Science (1996), Lyttle et al. (1996) Nucleic Acids Res. 24(14):2793, Shchepino et al. (2001) Nucleosides, Nucleotides, & Nucleic Acids 20:369, Doronina et al (2001) Nucleosides, Nucleotides, & Nucleic Acids 20:1007, Trawick et al. (2001) Bioconjugate Chem. 12:900, Olejnik et al. (1998) Methods in Enzymology 291:135, and Pljevaljcic et al. (2003) J. Am. Chem. Soc. 125(12):3486, all of which are incorporated by reference.

“Fragment” refers to a piece of contiguous nucleic acid that contains fewer nucleotides than the complete nucleic acid.

“Hybridization,” “annealing,” “selectively bind,” or “selective binding” refers to the base-pairing interaction of one nucleic acid with another nucleic acid (typically an antiparallel nucleic acid) that results in formation of a duplex or other higher-ordered structure (i.e. a hybridization complex). The primary interaction between the antiparallel nucleic acid molecules is typically base specific, e.g., A/T and G/C. It is not a requirement that two nucleic acids have 100% complementarity over their full length to achieve hybridization. Nucleic acids hybridize due to a variety of well characterized physio-chemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes part I chapter 2, “Overview of principles of hybridization and the strategy of nucleic acid probe assays,” (Elsevier, New York), as well as in Ausubel (Ed.) Current Protocols in Molecular Biology, Volumes I, II, and III, 1997, which is incorporated by reference.

“Nucleic acid” or “nucleic acid molecule” refers to a multimeric compound comprising two in more covalently bonded nucleosides or nucleoside analogs having nitrogenous heterocyclic bases, or base analogs, where the nucleosides are linked together by phosphodiester bonds or other linkages to form a polynucleotide. Nucleic acids include RNA, DNA, or chimeric DNA-RNA polymers or oligonucleotides, and analogs thereof. A nucleic acid backbone can be made up of a variety of linkages, including one or more of sugar-phosphodiester linkages, peptide-nucleic acid bonds, phosphorothioate linkages, methylphosphonate linkages, or combinations thereof. Sugar moieties of the nucleic acid can be ribose, deoxyribose, or similar compounds having known substitutions (e.g. 2′-methoxy substitutions and 2′-halide substitutions). Nitrogenous bases can be conventional bases (A, G, C, T, U) or analogs thereof (e.g., inosine, 5-methylisocytosine, isoguanine). A nucleic acid can comprise only conventional sugars, bases, and linkages as found in RNA and DNA, or can include conventional components and substitutions (e.g., conventional bases linked by a 2′-methoxy backbone, or a nucleic acid including a mixture of conventional bases and one or more base analogs). Nucleic acids can include “locked nucleic acids” (LNA), in which one or more nucleotide monomers have a bicyclic furanose unit locked in an RNA mimicking sugar conformation, which enhances hybridization affinity toward complementary sequences in single-stranded RNA (ssRNA), single-stranded DNA (ssDNA), or double-stranded DNA (dsDNA). Nucleic acids can include modified bases to alter the function or behavior of the nucleic acid (e.g., addition of a 3′-terminal dideoxynucleotide to block additional nucleotides from being added to the nucleic acid). Synthetic methods for making nucleic acids in vitro are well known in the art although nucleic acids can be purified from natural sources using routine techniques. Nucleic acids can be single-stranded or double-stranded.

An “oligonucleotide” or “oligomer” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleatide. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis.

Nucleases are enzymes that cleave the phosphodiester bond within a polynucleotide chain. Endonucleases include, but are not limited to, Cas proteins, restriction endonucleases, meganucleases, homing endonucleases, TAL effector nucleases, and Zinc finger nucleases. Endonucleases include, but are not limited to, Type I, Type II, Type III, Type IV, and Type V endonucleases, any one of which may further include subtypes. Cas proteins include, but are not limited to, Cas1, Cas1B, Cas2Cas3, Cas3′ (Cas3-prime), Cas3″ (Cas3-double prime), Cas4, Cas5, Cas6, Cas6e (formerly referred to as CasE, Cse3), Cas6f (i.e., Csy4), Cas7, Cas8, Cas8a1, Cas8a2, Cas8b, Cas8c, Cas9 (also known as Csn1 and Csx12), Cas10, Cas10d, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6Csb1, Csb2, Csb3, Csx17, Csx14, Csx10, Csx16, CsaX, Csx3, Csx1, Csx15, Csf1, Csf2, Csf3, Csf4, homologs thereof, and modified versions thereof. One skilled in the art can choose a nuclease based on various factors, including size, stability, ability to bind to a guide nucleic acid, ability to recognize a target sequence, etc. In some embodiments, the nuclease may be further optimized (e.g., to have a longer halflife, to be codon-optimized for the organism, to further comprise a nuclear localization signal, etc.). In some embodiments, the nuclease can be fused to other functional groups, for example a GFP domain, to visualize the protein.

In some embodiments, the nuclease may be Cas9. In some embodiments, the nuclease may be a Cas9 cloned or derived from a bacteria (S. pyogenes, S. pneumoniae, S. aureus, or S. thermophilus). One skilled in the art will recognize there are many Cas9 nucleases derived from bacteria. One skilled in the art can choose a Cas9 nuclease based on various factors, including size, stability, ability to bind to a guide nucleic acid, ability to recognize a protospacer adjacent motif (i.e., PAM) etc. In some embodiments, the Cas9 nuclease may be further optimized (e.g., to have a longer half-life, to be codon-optimized for the organism, to further comprise a nuclear localization signal, etc.). In some embodiments, the Cas9 nuclease can be fused to other functional groups, for example a GFP domain, to visualize the protein.

A Cas9 protein may recognize a protospacer adjacent motif (PAM) sequence comprising NGG. A Cas9 protein may recognize a protospacer adjacent motif (PAM) sequence that does not comprise NGG. A Cas9 protein may recognize a protospacer adjacent motif (PAM) sequence comprising NNGRRT, such as TTGAAT or TTGGGT.

An endonuclease may have DNA cleavage activity, such as Cas9. In some embodiments, an endonuclease directs cleavage of one or both strands at the location of a target sequence, such as within the target sequence and/or within the complement of the target sequence. In some embodiments, an endonuclease directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some embodiments, an endonuclease is mutated with respect to a corresponding wild-type enzyme such that the mutated endonuclease lacks the ability to cleave one or both strands of a target polynucleotide containing a target sequence. For example, an aspartate-to-alanine substitution (e.g., D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In some embodiments, the Cas protein (e.g., Cas9 protein) may be a nickase. In aspects of the invention, nickases may be used for genome editing via homologous recombination. In some embodiments, a Cas9 nickase may be used in combination with guide polynucleotide(s), e.g., two guide polynucleotides, which target respectively sense and antisense strands of the DNA target. Two or more catalytic domains of Cas9 (RuvC I, RuvC II, and RuvC III) may be mutated to produce a mutated Cas9 substantially lacking DNA cleavage activity. In some embodiments, a D10A mutation is combined with one or more of H840A, N854A, or N863A mutations to produce a Cas9 enzyme substantially lacking DNA cleavage activity. In some embodiments, an endonuclease is considered to substantially lacking DNA cleavage activity when the DNA cleavage activity of the mutated endonuclease is less than about 25%, 20%, 15%, 10%, 5%, 4%, 3%, 2%, 1%, 0.5%, 0.1%, 0.05%, 0.01%, or lower than 0.01% with respect to its non-mutated form.

In some embodiments, a gene encoding an endonuclease (e.g., a Cas protein such as Cas9) is codon optimized for expression in particular cells, such as eukarymic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cell of interest by replacing at least one codon (e.g., about or more than about 1, 2, 3, 4, 5, 10, 15, 20, 25, 50, or more than 50 codons) of the native sequence with codons that are more frequently or most frequently used in the genes of that host cell while maintaining the native amino acid sequence. Various species may exhibit particular bias for certain codons of a particular amino acid. Codon bias (differences in codon usage between organisms) may correlate with the efficiency of translation of messenger RNA (mRNA), which may depend on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. In some embodiments, one or more codons (e.g., 1, 2, 3, 4, 5, 10, 15, 20, 25, 50, more than 50, or all codons) in a sequence encoding an endonuclease correspond to the most frequently used codon for a particular amino acid. In certain embodiments, a gene encoding an endonuclease may not be codon optimized.

In some embodiments, an endonuclease is part of a fusion protein comprising one or more heterologous peptide or protein domains (e.g., about or at least about 1, 2, 3, 4, 5, 6, 7, 8, 9, 10, or more than 10 domains in addition to an endonuclease). An endonuclease fusion protein may comprise any additional peptide or protein sequence, and optionally a linker sequence between any two domains. Examples of peptide or protein domains that may be fused to an endonuclease include, without limitation, epitope tags, reporter gene sequences, localization signals, and protein domains having one or more of the following activities: methylase activity, demethylase activity, transcription activation activity, transcription repression activity, transcription release factor activity, histone modification activity, RNA cleavage activity, and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), other fluorescent proteins, and autofluorescent proteins including blue fluorescent protein (BFP). An endonuclease may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Examples of localization signals include, but are not limited to, nuclear localization signals (e.g., SV40 large T-antigen, acidic M9 domain of hnRNP A1), cytoplasmic localization signals, mitochondrial localization signals, nuclear export signals, chloroplast localization signals, and endoplasmic reticulum retention signals. In some embodiments, a tagged endonuclease is used to identify the location of a target sequence.

As used herein, the term “guide polynucleotide”, refers to a polynucleotide sequence that can form a complex with an endonuclease (e.g., Cas protein such as Cas9) and enables the endonuclease to recognize and optionally cleave a target site on a polynucleotide such as DNA. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond, or linkage modification such as, but not limited, to locked nucleic acid (LNA), peptide nucleic acid (PNA), bridged nucleic acid (BNA), 5-methyl dC, 2,6-Diaminopurine, 2′-Fluoro A, 2′-Fluoro U, 2′-O-Methyl RNA, Phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. In some embodiments, the guide polynucleotide does not solely comprise ribonucleic acids (RNAs). In other embodiments, the guide polynucleotide does solely comprise ribonucleic acids (RNAs). A guide polynucleotide that solely comprises ribonucleic acids is also referred to as a “guide RNA”.

The guide polynucleotide can be a double molecule (also referred to as duplex guide polynucleotide) comprising a first nucleotide sequence domain (referred to as Variable Targeting domain or VT domain) that is complementary to a nucleotide sequence in a target DNA and a second nucleotide sequence domain (referred to as Cas endonuclease recognition domain or CER domain) that interacts with a Cas endonuclease. The CER domain of the double molecule guide polynucleotide comprises two separate molecules that are hybridized along a region of complementarity. The two separate molecules can be RNA, DNA, and/or RNA-DNA combination sequences. In some embodiments, the duplex guide polynucleotide does not solely comprise ribonucleic acids (RNAs). In some embodiments, the first molecule of the duplex guide polynucleotide comprising a VT domain linked to a CER domain is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). In some embodiments, the second molecule of the duplex guide polynucleotide comprising a CER domain is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides).

The guide polynucleotide can also be a single molecule comprising a first nucleotide sequence domain (referred to as Variable Targeting domain or VT domain) that is complementary to a nucleotide sequence in a target DNA and a second nucleotide domain (referred to as endonuclease recognition domain or CER domain) that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. In some embodiments, the single guide polynueleotide comprises a crNucleotide (comprising a VT domain linked to a CER domain) linked to a tracrNucleotide (comprising a CER domain), wherein the linkage is a nucleotide sequence comprising a RNA sequence, a DNA sequence, or a RNA-DNA combination sequence. The single guide polynucleotide being comprised of sequences from the crNucleotide and tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides).

The term “variable targeting domain” or “VT domain” is used interchangeably herein and refers to a nucleotide sequence that is complementary to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

In general, a guide polynucleotide is any polynucleotide sequence having sufficient complementarity with a target polynucleotide sequence to hybridize with the target sequence and direct sequence-specific binding of a CRISPR complex to the target sequence. In some embodiments, the degree of complementarity between a guide polynucleotide and its corresponding target sequence, when optimally aligned using a suitable alignment algorithm, is about or more than about 50%, 55%, 60%, 65%, 70%, 75%, 80%, 85%, 90%, 95%, 96%, 97%, 98%, 99%, 99.5%, 99.9%, or 100%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide polynucleotide is about or at least about 5, 10, 11, 12, 13, 14, 15, 16, 17, 18, 19, 20, 21, 22, 23, 24, 25, 26, 27, 28, 29, 30, 35, 40, 45, 50, 75, or more than 75 nucleotides in length. In some embodiments, a guide polynucleotide is up to about 75, 50, 45, 40, 35, 30, 25, 20, 15, 12, or fewer than 12 nucleotides in length. The ability of a guide polynucleotide to direct sequence-specific binding of a CRISPR complex to a target sequence may be assessed by any suitable assay.

A guide polynucleotide may be selected to target any target sequence. In some embodiments, the target sequence is a sequence within a genome of a cell. Exemplary target sequences include those that are unique in the target genome.

A homology arm may be about 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, or more than 500 nucleotides in length. In some embodiments, homology arms on a construct are the same length, similar lengths, or different lengths. In some embodiments, the degree of complementarily between a homology arm and its corresponding target sequence, when optimally aligned using a suitable alignment algorithm, is about or more than about 50%, 55%, 60%, 65%, 70%, 75%, 80%, 85%, 90%, 95%, 96%, 97%, 98%, 99%, 99.5%, 99.9%, or 100%. In some instances, the homology arms directly abut the endonuclease cleavage sites. In some embodiments of any one of the methods or constructs described herein, the homology arms directly abut the endonuclease cut site(s) determined by the at least one guide polynucleotide, or are separated by less than 100, 75, 50, 40, 30, 20, 10, 9, 8, 7, 6, 5, 4, 3, 2, or 1 nucleic acids.

A cell has been “genetically modified” or “transformed” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication.

Methods of introducing a nucleic acid into a host cell are known in the art, and any known method can be used to introduce a nucleic acid (e.g., an expression construct) into a cell. Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, direct micro injection, nanoparticle-mediated nucleic acid delivery (see, e.g., Panyam et., al Adv Drug Deliv Rev. 2012 Sep. 13, pii: S0169-409X(12)00283-9, doi: 10.1016/j.addr.2012.09.023), and the like.

Similar strategies can be devised to combat other insect borne diseases. Insects that carry insect borne diseases include, but are not limited to, the mosquito, tick, flea, lice, Culicoid midge, sandfly, Tsetse fly, and bed bug. Insect borne diseases include, but are not limited to, mosquito borne diseases, tick borne diseases, flea borne diseases, lice borne diseases, Culicoid midge borne diseases, sandfly home diseases, Tsetse fly borne diseases, bed bug borne diseases, and any combination thereof. Examples of insect borne diseases include, but are not limited to, African horse sickness, babesiosis, bluetongue disease, tick-borne encephalitis, Rickettsial diseases (e.g., typhus, rickettsialpox, Boutonneuse fever, African tick bite fever, Rocky Mountain spotted fever), Crimean-Congo hemorrhagic fever, ehrlichiosis, Southern tick-associated rash illness, tick-borne relapsing fever, tularemia, lice infestation, heartland virus, plague, Trypanosomiasis, sleeping sickness, leishmaniasis, Chagas disease, and Lyme disease. Mosquito borne diseases include, but are not limited to, malaria, dengue fever, yellow fever, chikungunya, dog heartworm, Eastern equine encephalitis, epidemic polyarthritis, filariasis, Rift Valley fever, Ross River fever, St. Louis encephalitis, Japanese encephalitis, pogosta disease, LaCrosse encephalitis, Western equine encephalitis, and West Nile virus.

Treating diseases or conditions: Elements can be designed that treat diseases or conditions by selectively adding, deleting, or mutating genes. For example, genes that encode immunogenic proteins may be targeted to reduce or eliminate immunogenicity. Allergens in food may be reduced by targeting the genes encoding the allergen in the organism (e.g., peanut, tree nut, cow (or other source of milk), chicken (or other source of egg), wheat, soy, fish, shellfish) from which the food was derived. Specific cells may be targeted, such as beta cells (role in diabetes) or cells and/or genes involved in autoimmune disorders.

Controlling Agriculture Pest Species: Agriculture pests and invasive species cause over $3 billion of damage to crops per year. Constructs targeting one or more genes, for example those required for female fertility or survival, may reduce the damage caused by many of these pests.

For instance, the invention can suppress crop pests actively attacking a crop of interest or be used for weed control. This strategy closely parallels that illustrated above for combating malaria. For example, the spotted wing fly (Drosophila suzukii), which is related to the laboratory fruit fly (Drosophila melanogaster), may be targeted. D. suzukii entered the U.S. in 2008 and in 2010 was estimated to cause over $500 million of damage is to soft fruits (strawberries, other berries, grapes, cherries) in Pacific coast states, amounting to nearly 20% of this $2.5 billion industry. The genome sequence of D. suzukii has been determined, and constructs described herein can be generated to test for control and eradication of this invasive pest. Other pests that may also be targeted include, but are not limited to, the Medfly (≈$1.2 billion damage/yr), olive fly (can reduce oil production by as much as 80%), pea leaf miner (a fly causing over $1.5 billion of crop damage), and Asian tiger mosquito (a vector for encephalitis, dengue fever, yellow fever and dog heartworm). Pests or weeds that are resistant to pesticides or herbicides (e.g., glyphosate), respectively, may also be targeted by the invention. For example, allelic-drive elements may replace resistant alleles to restore susceptibility to a pesticide or herbicide. Resistant pests that may be targeted include, but are not limited to, the western corn rootworm, horseweed, pigweed, Amaranthus hybridus (syn: quitensis) (Smooth Pigweed); Amaranthus palmeri (Palmer Amaranth); Amaranthus spinosus (Spiny Amaranth); Amaranthus tuberculatus (=A, rudis) (Tall Waterhemp); Ambrosia artemisiifolia (Common Ragweed); Ambrosia trifida (Giant Ragweed); Bidens pilosa (Hairy Beggarticks); Brachiaria eruciformis (Sweet Summer Grass); Bromus diandrus (Ripgut Brome); Bromus rubens (Red Brome); Chloris elata (Tall Windmill Grass); Chloris truncata (Windmill Grass); Conyza bonariensis (Hairy Fleabane); Conyza canadensis (Horseweed); Conyza sumatrensis (Sumatran Fleabane); Cynodon hirsutus (Gramilla mansa); Digitaria insularis (Sourgrass); Echinochloa colona (Junglerice); Eleusine indica (Goosegrass); Hedyotis verticillata (Woody borreria); Kochia scoparia (Kochia); Leptochloa virgata (Tropical Sprangletop, Juddsgrass); Lolium perenne (Perennial Ryegrass); Lolium perenne ssp. multiflorum (Italian Ryegrass); Lolium rigidum (Rigid Ryegrass); Parthenium hysterophorus (Ragweed Parthenium); Plantago lanceolata (Buckhorn Plantain); Poa annua (Annual Bluegrass); Raphanus raphanistrum (Wild Radish); Sonchus oleraceus (Annual Sowthistle); Sorghum halepense (Johnsongrass); Urochloa panicoides (Liverseedgrass) and any combination thereof. By reducing resistance or reversing it, a pesticide or herbicide may be used for a longer period of time and/or in lower concentrations or amounts.

Agriculture pests include, but are not limited to, agriculture pest insects, agriculture pest mites, agriculture pest nematodes, grape pests, pest molluscs, strawberry pests, Western honey bee pests, insect pests of ornamental plants, insect vectors of plant pathogens, plant pathogenic nematodes, invasive species, and any combination thereof.

Agriculture pest insects include, but are not limited to, Acalymma, Acrythosiphon kondoi, Acyrthosiphon gossypii, Acyrthosiphon pisum, African armyworm, Africanized bee, Agrilus planipennis (Emerald ash borer), Agromyzidae, Agrotis ipsilon, Agrotis munda, Agrotis porphyricollis, Akkaia taiwana, Aleurocanthus woglumi, Aleyrodes proletella, Alphitobius diaperinus, Alsophila aescularia, Altica chalybea, Ampeloglypter ater, Anasa tristis, Anisoplia austriaca, Anthonomus pomorum, Anthonomus signatus, Aonidiella aurantii, Apamea apamiformis, Apamea niveivenosa, Aphid, Aphis gossypii, Aphis nasturtii, Apple maggot, Argentine ant, Army cutworm, Arotrophora arcuatalis, Astegopteryx bambusae, Astegopteryx insularis, Astegopteryx minuta, Asterolecanium coffeae, Atherigona reversura, Athous haemorrhoidalis, Aulacophora, Aulacorthum solani, Australian plague locust, Bactericera cockerelli, Bactrocera, Bactrocera correcta, Bagrada hilaris, Beet armyworm, Black bean aphid, Blepharidopterus chlorionis, Bogong moth, Boll weevil, Bollworm, Brassica pod midge, Brevicoryne brassicae, Brown locust, Brown marmorated stink bug, Brown planthopper Cabbage moth, Cabbage worm, Callosobruchus maculatus, Cane beetle, Carrot fly, Cerataphis brasiliensis, Ceratitis capitata, Ceratitis rosa, Ceratoglyphina bambusae, Ceratopemphigus zehntneri, Ceratovacuna lanigera, Cereal leaf beetle, Chaetosiphon tetrarhodum, Chlorops pumilionis, Chrysophtharta bimaculata, Citrus flatid planthopper, Citrus long-horned beetle, Coccus hesperidum, Coccus viridis, Codling moth, Coffee borer beetle, Colorado potato beetle, Confused flour beetle, Crambus, Cucumber beetle, Curculio nucum, Curculio occidentis, Cutworm, Cyclocephala borealis, Date stone beetle, Delia (genus), Delia antiqua, Delia floralis, Delia radicum, Desert locust, Diabrotica, Diabrotica balteata, Diabrotica speciosa, Diamondback moth, Diaphania indica, Diaphania nitidalis, Diaphorina citri, Diaprepes abbreviatus, Diatraea saccharalis, Differential grasshopper, Dociostaurus maroccanus, Drosophila suzukii, Dryocosmus kuriphilus, Dysaphis crataegi, Earias perhuegeli, Epicauta vittata, Epilachna varivestis, Erionota thrax, Eriosoma lanigerum, Eriosomatinae, Euleia heraclei, Eumetopina flavipes, Eupoecilia ambiguella, European corn borer, Eurydema oleracea, Eurygaster integriceps, Ferrisia virgata, Forest bug, Frankliniella tritici, Galleria mellonella, Garden Dart, Geoica lucifuga, Glassy-winged sharpshooter, Great French Wine Blight, Greenhouse whitefly, Greenidea artocarpi, Greenidea formosana, Greenideoida ceyloniae, Gryllotalpa orientalis, Gypsy moths in the United States, Helicoverpa armigera, Helicoverpa gelotopoeon, Helicoverpa punctigera, Helicoverpa zea, Heliothis virescens, Henosepilachna vigintioctopunctata, Hessian fly, Hyalopterus pruni, Hysteroneura setariae, Ipuka dispersum, Jacobiasca formosana, Japanese beetle, Kaltenbachiella elsholtriae, Kaltenbachiella japonica, Khapra beetle, Knulliana, Lampides boeticus, Leaf miner, Leek moth, Lepidiota consobrina, Lepidosaphes beckii, Lepidosaphes ulmi, Leptocybe, Leptoglossus zonatus, Leptopterna dolabrata, Lesser wax moth, Leucoptera (moth), Leucoptera caffeina, Light brown apple moth, Light brown apple moth controversy, Lipaphis erysimi, Lissothoptrus oryzophilus, Long-tailed skipper, Lygus, Lygus hesperus, Maconellicoccus hirsutus, Macrodactylus subspinosus, Macrosiphoniella pseudoartemisiae, Macrosiphoniella sanborni, Macrosiphum euphorbiae, Maize weevil, Manduca sexta, Matsumuraja capitophoroides, Mayetiola hordei, Mealybug, Megacopta cribraria, Melanaphis sacchari, Micromyzus judenkoi, Micromyzus kalimpongensis, Micromyzus niger, Moth, Myzus ascalonicus Myzus boehmeriae, Myzus cerasi, Myzus obtusirostris, Myzus omatus, Myzus persicae, Neomyzus circumflexus, Neotoxoptera oliveri, Nezara viridula, Nomadacris succincta, Oak processionary, Oebalus pugnax, Olive fruit fly, Ophiomyia simplex, Opisina arenosella, Opomyza, Opomyza florum, Opomyzidae, Oscinella frit, Ostrinia furnacalis, Oxycarenus hyalinipennis, Papilio demodocus, Paracoccus marginatus, Paralobesia viteana, Paratachardina pseudolobata, Pentalonia nigronervosa, Pentatomoidea, Phorodon humuli, Phthorimaea operculella, Phyllophaga, Phylloxeridae, Phylloxeroidea, Pieris brassicae, Pink bollworm, Planococcus citri, Platynota idaeusalis, Plum curculio, Prionus californicus, Pseudococcus maritimus, Pseudococcus viburni, Pseudoregma bambucicola, Pyralis farinalis, Red imported fire ant, Red locust, Rhagoletis cerasi, Rhagoletis indifferens, Rhagoletis mendax, Rhodobium porosum, Rhopalosiphoninus latysiphon, Rhopalosiphum maidis, Rhopalosiphum padi, Rhopalosiphum rufiabdominale, Rhyacionia frustrana, Rhynchophorus ferrugineus, Rhynchophorus palmarum, Rhyzopertha, Rice moth, Russian wheat aphid, San Jose scale, Scale insect, Schistocerca americana, Schizaphis graminum, Schizaphis hypersiphonata, Schizaphis minuta, Schizaphis rotundiventris, Schoutedenia lutea, Sciaridae, Scirtothrips dorsalis, Scutelleridae, Scutiphora pedicellata, Serpentine leaf miner, Setaceous Hebrew character, Shivaphis celti, Silver Y, Silverleaf whitefly, Sinomegoura citricola, Sipha nava, Sitobion avenae, Sitobion lambersi, Sitobion leelamaniae, Sitobion miscanthi, Sitobion pauliani, Sitobion phyllanthi, Sitobion wikstroemiae, Small hive beetle, Southwestern corn borer, Soybean aphid, Spodoptera cilium, Spodoptera litura, Spotted cucumber beetle, Squash vine borer, Stemborer, Stenotus binotatus, Strauzia longipennis, Striped flea beetle, Sunn pest, Sweetpotato bug, Synanthedon exitiosa, Tarnished plant bug, Tetraneura nigriabdominalis, Tetraneura yezoensis, Thrips, Thrips angusticeps, Thrips palmi, Tinocallis kahawaluokalani, Toxoptera aurantii, Toxoptera citricida, Toxoptera odinae, Trioza erytreae, Turnip moth, Tuta absoluta, Uroleucon minutum, Varied carpet beetle, Vesiculaphis caricis, Virachola isocrates, Waxworm, Western corn rootworm, Western flower thrips, Wheat fly, Wheat weevil, Whitefly, Winter moth, Xylotrechus quadripes, and any combination thereof.

Agriculture pest mites include, but are not limited to, Abacarus hystrix, Abacarus sacchari, Acarapis woodi, Aceria guerreronis, Aceria tosichella, Brevipalpus phoenicis, Dermanyssus gallinae, Eriophyes padi, Eriophyidae, Flour mite, Oligonychus sacchari, Panonychus ulmi, Polyphagotarsonemus latus, Redberry mite, Steneotarsonemus spinki, Tetranychus urticae, Tuckerella, Varroa destructor, Varroa jacobsoni, Varroa sensitive hygiene, and any combination thereof.

Agriculture pest nematodes include, but are not limited to, Achlysiella williamsi, Anguina (nematode), Anguina agrostis, Anguina amsinckiae, Anguina australis, Anguina balsamophila, Anguina funesta, Anguina graminis, Anguina spermophaga, Anguina tritici, Aphelenchoides, Aphelenchoides arachidis, Aphelenchoides besseyi, Aphelenchoides fragariae, Aphelenchoides parietinus, Aphelenchoides ritzemabosi, Aphelenchoides subtenuis, Belonolaimus, Belonolaimus gracilis, Belonolaimus longicaudatus, Cereal cyst nematode, Coffee root-knot nematode, Ditylenchus, Ditylenchus africanus, Ditylenchus angustus, Ditylenchits destructor, Ditylenchus dipsaci, Dolichodorus heterocephalus, Fig Pin Nematode, Foliar nematode, Globodera pallida, Globodera rostochiensis, Globodera tabacum, Helicotylenchus dihystera, Hemicriconemoides kanayaensis, Hemicriconemoides mangiferae, Hemicycliophora arenaria, Heterodera avenae, Heterodera cajani, Heterodera carotae, Heterodera ciceri, Hoplolaimus galeatus, Hoplolaimus indicus, Hoplolaimus magnistylus, Hoplolaimus seinhorsti, Hoplolaimus uniformis, Longidorus africanus, Longidorus maximus, Longidorus sylphus, Meloidogyne acronea, Meloidogyne arenaria, Meloidogyne artiellia, Meloidogyne brevicauda, Meloidogyne chitwoodi, Meloidogyne enterolobii, Meloidogyne incognita, Meloidogyne javanica, Meloidogyne naasi, Meloidogyne partityla, Meloidogyne thamesi, Merlinius brevidens, Mesocriconema xenoplax, Nacobbus aberrans, Northern root-knot nematode, Paralongidorus maximus, Paratrichodorus minor, Paratylenchus curvitatus, Paratylenchus elachistus, Paratylenchus macrophallus, Paratylenchus microdorus, Paratylenchus projectus, Paratylenchus tenuicaudatus, Potato cyst nematode, Pratylenehus alleni, Quinisulcius acutus, Quinisulcius capitatus, Radopholus similis, Soybean cyst nematode, Tylenchorhynchus, Tylenchorhynchus brevilineatus, Tylenchorhynchus claytoni, Tylenchorhynchus dubius, Tylenchorhynchus maximus, Tylenchorhynchus nudes, Tylencharhynchus phaseoli, Tylenchorhynchus vulgaris, Tylenchorhynchus zeae, Tylenchulus semipenetrans, Xiphinema, Xiphinema americanum, Xiphinema bakeri, Xiphinema brevicolle, Xiphinema diversicaudatum, Xiphinema insigne, Xiphinema rivesi, Xiphinema Vuittenezi, and any combination thereof.

Grape pests include, but are not limited to, Ampeloglypter ater, Ampeloglypter sesostris, Eriophyes vitis, Eupoecilia ambiguella, Fig Pin Nematode, Great French Wine Blight, Japanese beetle, List of Lepidoptera that feed on grapevines, Maconellicoccus hirsutus, Mesocriconema xenoplax, Otiorhynchus cribricollis, Paralobesia viteana, Paratrichodorus minor, Phylloxera, Pseudococcus maritimus, Pseudococcus viburni, Tetranychus urticae, Xiphinema index, Zenophassus, and any combination thereof.

Pest molluscs include, but are not limited to, Cornu aspersum, Deroceras, Grove snail, Limax, Milax gagates, Theba pisana, and any combination thereof.

Strawberry pests include, but are not limited to, Anthonomus rubi, Anthonomus signatus, Aphelenchoides fragariae, Otiorhynchus ovatus, Pratylenchus coffeae, Xiphinema diversicaudatum, and any combination thereof.

Western honey bee pests include, but are not limited to, Acarapis woodi, American foulbrood, Braula, Deformed wing virus, List of diseases of the honey bee, Nosema apis, Small hive beetle, Varroa destructor, Waxworm, and any combination thereof.

Insect pests of ornamental plants include, but are not limited to, Acieris variegana, Acyrthosiphon pisum, Alsophila aescularia, Aphid, Bird-cherry ermine, Coccus hesperidan, Coccus viridis, Contarinia quinquenotata, Grapeleaf skeletonizer, Gypsy moths in the United States, Japanese beetle, Macrodactylus subspinosus, Mealybug, Mullein moth, Orchidophilus, Otiorhynchus sulcatus, Paratachardina pseudolobata, Paysandisia archon, Sawfly, Scale insect, Scarlet lily beetle, Sciaridae, Spodoptera cilium, Stephanitis takeyai, Tenthredo scrophulariae, Yponomeuta malinellus, Yponomeuta padella, and any combination thereof.

Insect vectors of plant pathogens include, but are not limited to, Acyrthosiphon pisum, Agromyzidae, Authomyiidae, Aphid, Bark beetle, Beet leafhopper, Brevicoryne brassicae, Cacopsylla melanoneura, Chaetosiphon fragaefolii, Cicadulina, Cicadulina mbila, Common brown leafhopper, Cryptococcus fagisuga, Curculionidae, Diabrotica balteata, Empoasca decedens, Eumetopina flavipes, Euscelis plebejus, Frankliniella tritici, Glassy-winged sharpshooter, Haplaxius crudus, Hyalesthes obsoletus, Hylastes ater, Jumping plant louse, Leaf beetle, Leafhopper, Macrosteles quadrilineatus, Mealybug, Melon fly, Molytinae, Pegomya hyoscyami, Pissodes, Pissodes strobi, Pissodini, Planthopper, Pseudococcus maritimus, Pseudococcus viburni, Psylla pyri, Rhabdophaga rosaria, Rhynchophorus palmarum, Scaphoideus titanus, Scirtothrips dorsalis, Silverleaf whitefly, Tephritidae, Thripidae, Thrips palmi, Tomicus piniperda, Toxoptera citricida, Treehopper, Triozidae, Western flower thrips, Xyleborus glabratus, and any combination thereof.

Plant pathogenic nematodes include, but are not limited to, Helicotylenchus, Heterodera, Heterodera amygdali, Heterodera arenaria, Heterodera aucklandica, Heterodera bergeniae, Heterodera hi fenestra, Heterodera cacti, Heterodera canadensis, Heterodera cardiolata, Heterodera cruciferae, Heterodera delvii, Heterodera elachista, Heterodera filipjevi, Heterodera gambiensis, Heterodera goettinuiana, Heterodera hordecalis, Heterodera humuli, Heterodera latipons Heterodera medicaginis, Heterodera oryzae, Heterodera oryzicola, Heterodera rosii, Heterodera sacchari, Heterodera schachtii, Heterodera tabacum, Heterodera trifolii, Heteroderidae, Hirschmanniella oryzae, Hoplolaimidae, Hoplolaijus columbus, Hoplolaimus pararobustus, Meloidogyne fruglia, Meloidogyne gajuscus, Nacobbus dorsalis, Pratylenchus brachyurus, Pratylenchus coffeae, Pratylenchus crenatus, Pratylenchus dulscus, Pratylenchus fallax, Pratylenchus flakkensis, Pratylenchus goodeyi, Pratylenchus hexincisus, Pratylenchus loosi, Pratylenchus minutus, Pratylenchus mulchandi, Pratylenchus musicola, Pratylenchus neglectus, Pratylenchus penetrans, Pratylenchus pratensis, Pratylenchus reniformia, Pratylenchus scribneri, Pratylenchus thornei, Pratylenchus vulnus, Pratylenchus zeae, Punctodera chalcoensis, Root gall nematode, Root invasion (parasitic), Root-knot nematode, Rotylenchulus, Rotylenchulus parvus, Rotylenchulus reniformis, Rotylenchus brachyurus, Rotylenchus robustus, Scutellonema brachyurum, Scutellonema cavenessi, Subanguina radicicola, Subanguina wevelli, and any combination thereof.

Invasive species include, but are not limited to, Acacia mearnsii, Achatina fulica, Acridotheres tristis, Aedes albopictus, Anopheles quadrimaculatus, Anoplolepis gracilipes, Anoplophora glabripennis, Aphanomyces astaci, Ardisia elliptica, Arundo donax, Asterias amurensis, Banana bunchy top virus (BBTV), Batrachochytrium dendrobatidis, Bemisia tabaci, Boiga irregularis, Bufo marinas=Rhinella marina, Capra hircus, Carcinus maenas, Caulerpa taxifolia, Cecropia peltata, Cercopagis pengoi, Cervus elaphus, Chromolaena odorata, Cinara cupressi, Cinchona pubescens, Clarias batrachus, Clidemia hirta, Coptotermes formosanus, Corbula amurensis, Cryphonectria parasitica, Cyprinus carpio, Dreissena polymorpha, Eichhornia crassipes, Eleutherodactylus coqui, Eriocheir sinensis, Euglandina rosea, Euphorbia esula, Fallopia japonica=Polygonum cuspidatum, Felis catus, Gambusia affinis, Hedychium gardnerianum, Herpestes javanicus, Hiptage benghalensis, Imperata cylindrica, Lantana camara, Lates niloticus, Leucaena leucocephala, Ligustrum robustum, Linepithema humile, Lymantria dispar, Lythrum salicaria, Macaca fascicularis, Melaleuca quinquenervia, Miconia calvescens, Micropterus salmoides, Mikania micrantha, Mimosa pigra, Mnemiopsis leidyi, Mus musculus, Mustela erminea, Myocastor coypus, Morella faya, Mytilus galloprovincialis, Oncorhynchus mykiss, Ophiostoma ulmi sensu lato, Opuntia stricta, Oreochromis mossambicus, Oryctolagus cuniculus, Pheidole megacephala, Phytophthora cinnamomi, Pinus pinaster, Plasmodium relictum, Platydemus manokwari, Pomacea canaliculata, Prosopis glandulosa, Psidium cattleianum, Pueraria montana var. lobata, Pycnonotus cafer, Rana catesbeiana, Rattus rattus, Rubus ellipticus, Salmo trutta, Salvinia molesta, Schinus terebinthifolius, Sciurus carolinensis, Solenopsis invicta, Spartina anglica, Spathodea campanulata, Sphagneticola trilobata, Sturnus vulgaris, Sus scrofa, Tamarix ramosissima, Trachemys scripta elegans, Trichosurus vulpecula, Trogoderma granarium, Ulex europaeus, Undaria pinnatifida, Vespula vulgaris, Vulpes vulpes, Wasmannia auropunctata, and any combination thereof.

Similar methods may be used to generate libraries of model organisms; generate specific strains, breeds, or mutants of a model organism; for one-step mutagenesis schemes to generate scoreable recessive mutant phenotypes in a single generation; facilitate basic genetic manipulations in diverse experimental and agricultural organisms (e.g., accelerating the generation of combinatorial mutants and facilitating mutagenesis in polyploid organisms); accelerate genetic manipulations in animals (e.g., primates) or plants (e.g., trees) with a long generation time; and for gene therapy.

Model organisms include, but are not limited to, viruses, prokaryotes, eukaryates, protists, fungi, plants, invertebrate animals, vertebrate animals, and any combination thereof. A model organism may include, but is not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof.

Virus model organisms include, but are not limited to, Phage lambda; Phi X 174; SV40; T4 phage; Tobacco mosaic virus; Herpes simplex virus; and any combination thereof.

Prokaryotic model organisms include, but are not limited to, Escherichia coli; Bacillus subtilis; Caulobacter crescentus; Mycoplasma genitalium; Aliivibrio fischeri; Synechocystis; Pseudomonas fluorescens; and any combination thereof.

Protist model organisms include, but are not limited to, Chlamydomonas reinhardtii; Dictyostelium discoideum; Tetrahymena thermophila; Emiliania huxleyi; Thalassiosira pseudonana; and any combination thereof.

Fungal model organisms include, but are not limited to, Ashbya gossypii; Aspergillus nidulans; Coprinus cinereus; Cryptococcus neoformans; Cunninghamella elegans; Neurospora crassa; Saccharomyces cerevisiae; Schizophyllum commune; Schizosaccharomyces pombe; Ustilago maydis; and any combination thereof.

Plant model organisms include, but are not limited to, Arabidopsis thaliana; Boechera; Selaginella moellendorffii; Brachypodium distachyon; Setaria viridis; Lotus japonicus; Lemna gibba; Maize (Zea mays L.); Medicago truncatula; Mimulus guttatus; Nicotiana benthamiana; Nicotiana tabacum; Rice (Oryza sativa); Physcomitrella patens; Marchantia polymorpha; Populus; and any combination thereof.

Invertebrate animal model organisms include, but are not limited to, Amphimedon queenslandica; Arbacia punctulata; Aplysia; Branchiostoma floridae; Caenorhabditis elegans; Caledia captiva (Orthoptera); Callosobruchus maculatus; Chorthippus parallelus; Ciona intestinalis; Daphnia spp.; Coelopidae; Diopsidae; Drosophila (e.g., Drosophila melanogaster); Euprymna scolopes; Galleria mellonella; Gryllus bimaculatus; Hydra; Loligo pealei; Macrostomum lignano; Mnemiopsis leidyi; Nematostella vectensis; Oikopleura dioica; Oscarella carmela; Parhyale hawaiensis; Platynereis dumerilii; Podisma spp.; Pristionchus pacificus; Scathophaga stercoraria; Schmidtea mediterranea; Stomatogastric ganglion; Strongylocentrotus purpuratus; Symsagittifera roscoffensis; Tribolium castaneum; Trichoplax adhaerens; Tubifex tubifex; and any combination thereof.

Vertebrate animal model organisms include, but are not limited to, Laboratory mice; Bombina bombing, Bombina variegata; Cat (Felis sylvestris catus); Chicken (Gallus gallus domesticus); Cotton rat (Sigmodon hispidus); Dog (Canis lupus familiaris); Golden hamster (Mesocricetus auratus); Guinea pig (Cavia porcellus); Little brown bat (Myotis lucifugus); Medaka (Oryzias latipes, or Japanese ricefish); Mouse (Mus musculus); Poecilia reticulata; Rat (Rattus norvegicus); Rhesus macaque (or Rhesus monkey) (Macaca mulatta); Sea lamprey (Petromyzon murinus); Takifugu (Takifugu rubripes); Xenopus tropicalis; Xenopus laevis; Zebra finch (Taeniopygia guttata); Zebrafish (Danio rerio); African Killifish (Nothobranchius furzeri); Human (Homo sapiens); and any combination thereof.

### EXAMPLES

### Results

### Allele-Specific Cas9-Dependent Cleavage

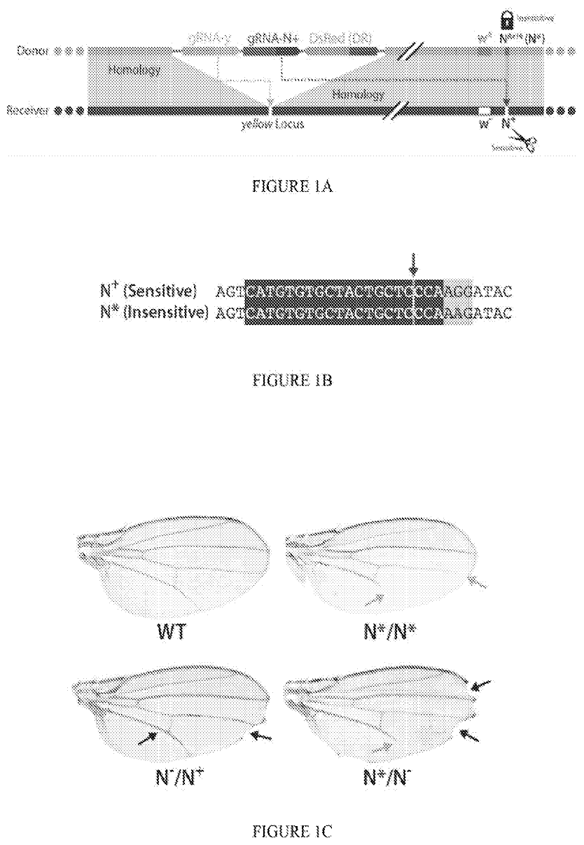

The X-linked Drosophila Notch (N) locus is particularly well suited for testing the allelic-drive of this invention since both loss- and gain-of-function dominant alleles of this locus have been characterized. Loss-of-function N⁻ mutations, which are non-viable when homozygous in females (or hemizygous in males), produce dominant wing margin notching and thickened veins phenotypes in heterozygous females (FIG. 1C, black arrows), while homozygous viable dominant gain-of-function alleles, designated as Abruptex (N^{Ax}, denoted as N* in the figures) generate a contrasting vein-loss phenotype (FIG. 1C, gray arrows).

Specifically, FIG. 1A shows a scheme depicting a DsRed-marked y^{<ccN>} CopyCat element⁷ that carries two gRNAs: 1) gRNA-y (yellow) (all color references are to grayscales), which drives copying of the y^{<ccN>} element at the yellow locus; and 2) gRNA-N+ (blue), which directs cleavage of the sensitive (S) wild-type Notch allele N^{+S} (scissors icon) to drive Super-Mendelian inheritance of the cleavage-insensitive (IS) N^{Ax16} allele (N^{*IS}, lock icon). Cas9 is provided in trans. FIG. 1B shows a DNA sequence of the gRNA-N+ target site on the sensitive wild-type Notch allele (N^{+S}), and the cleavage-insensitive N^{Ax16} allele (N^{*IS}) is highlighted in dark gray, and PAM site in light gray. The critical G→A substitution in the PAM site conferring cleavage insensitivity in N^{Ax16} mutants is bolded. The Cas9/gRNA cleavage site is indicated with a dashed line. FIG. 1C shows wing phenotypes of wild-type (WT), N^{Ax16} homozygous (N^{*}/N^{*}), N⁻ loss-of-function heterozygous (N⁻/N⁺), and N^{Ax16}/N⁻ heterozygous (N⁺/N⁻) Drosophila adults. FIG. 1D shows a crossing scheme used to generate F1 “master females” and genotype classes of 104 isogenic lines from single F2 females (detailed analysis in Table 2). X donor chromosome carrying the DsRed marked y^{<ccN>} element (DR) and the N^{Ax16} (N*) allele appears in light gray. WT (++) cut sensitive receiver chromosome is in dark gray. Third chromosome carrying a GFP-marked transgene expressing Cas9 (vasaCas9) and wild-type (+) chromosomes appear in light grey. The multiply inverted FM7 balancer chromosome is depicted in medium grey. Arrowheads indicate copying of the y^{<ccN>} element and the N^{Ax16} allele, respectively. FIG. 1E shows a percent transmission of y^{<ccN>} (DR, red circles—clustered around 85.3% and 72.4%) and N^{Ax16} allele (N^{*}, blue circles—clustered around 93.6%) in the presence or absence (grey circles) of Cas9. p-value intervals for this and all subsequent unpaired parametric t-test analysis: ****=p<0.0001; **=p<0.01; *=p<0.05. Bars denote mean value and standard deviation in this and all subsequent graphs. FIG. 1F shows a percent conversion of receiver chromosomes in F2 progeny from F1 master females (y^{<ccN>} w³ N^{Ax16}/++; Cas9/+ ♀ X w⁻ ♂). Eye color was used to distinguish progeny receiving donor (w³=orange eyes) versus receiver (w⁻=white eyes) chromosomes.

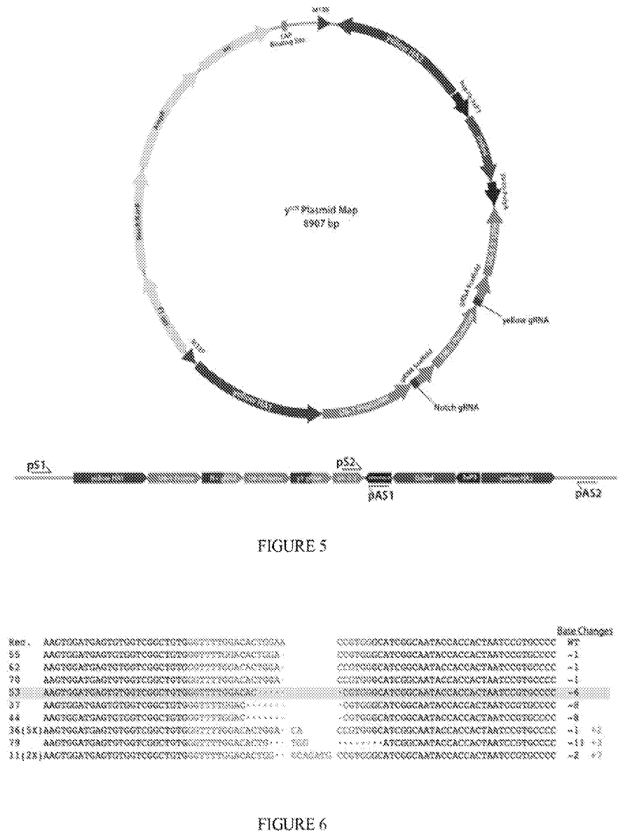

One previously identified N^{Ax16} allele (Ax16) eliminates a PAM site present on the wild-type allele (FIG. 1B, light gray box) resulting in a Gly→Arg amino acid substitution¹¹ (FIG. 10).

Specifically, FIG. 10 shows sequences of genomic DNA mutations and resulting amino acid alterations in various NHEJ-induced Notch alleles. The two N⁺ Insensitive alleles found cause the following amino-acid substitutions: Ser→Tyr for the N^{+IS1} mutation, and Ser Gln→Tyr Ala for the composite N^{+IS2} mutation. Note that six de novo NHEJ induced Abruptex alleles (including N^{Ax11} and N^{Ax103}) delete one amino acid, the N^{Ax26} allele results in the insertion of a single Tyr residue, and the N^{Ax3} allele results in the insertion of two amino-acids (Thr-Ser). NHEJ-induced N⁻ mutations cause deletions ranging from two to six amino-acids.

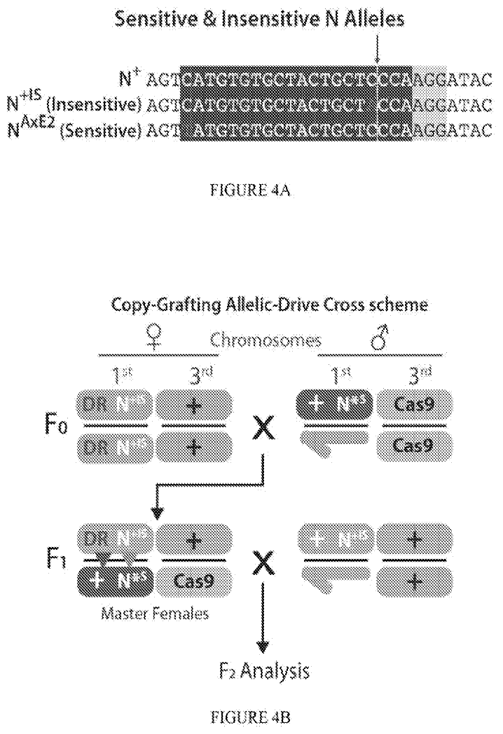

A gRNA (gRNA-N+) anchored by this PAM site was designed to direct cleavage of the wild-type N⁺ allele, but not the N^{Ax16} allele (FIGS. 1A-1B). The gRNA-N+ was incorporated into a DsRed-marked gRNA-only “CopyCat” element (ccN) designed to insert into, and copy itself, at the closely linked yellow locus, which is located 2 centimorgans distal to Notch relative to the centromere (FIG. 1A, FIG. 8B). Since genomic insertion of the ccN CopyCat element disrupts the function of the yellow locus this allele is denoted as y^{<ccN>} (see Methods for full allele nomenclature). lt was hypothesized that in the presence of an unlinked source of Cas9, this DsRed marked y^{<ccN>} CopyCat element would copy itself at the yellow locus and might also result in Super-Mendelian inheritance of the gRNA-insensitive N^{Ax16} allele via copy-cutting.

Whether the y^{<ccN>} allele would efficiently copy itself, as well as the neighboring uncleavable N^{Ax16} allele, onto a wild-type (y⁺ N⁺) X-chromosome (FIG. 1A) when combined with an autosomal source of Cas9 provided in-trans (FIG. 1D) was tested. F0 females (♀) bearing the y^{<ccN>} and N^{Ax16} alleles were crossed to wild-type males (♂) homozygous for a Cas9 source on the third chromosome to generate F1 y^{<ccN>} N^{Ax16}/++; Cas9/+ ♀ progeny, hereafter referred to as “master females” (abbreviated MF in figures)”. In parallel, F0 were crossed to w⁻ ♂ (lacking Cas9), to generate control F1 females (y^{<ccN>} N^{Ax16}/++; +/+ ♀), which were used to assess baseline Mendelian inheritance in the absence of Cas9-mediated drive. F1 master females (and control females) were crossed to wild-type (y⁺ N⁺) ♂ carrying a normal X-chromosome (or the multiply inverted FM7 balancer chromosome, which suppresses recombination, FIG. 1D). The resulting F2 progeny were then scored both for transmission of the DsRed-marked y^{<ccN>} element and the N^{Ax16} allele.

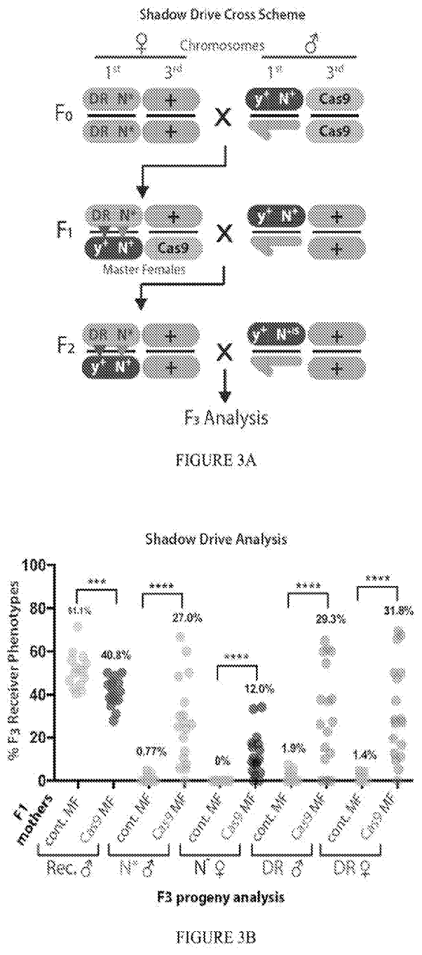

Transmission percentages for the y^{<ccN>} (DsRed+) and dominant N^{Ax16} alleles in F2 ♂ revealed highly biased inheritance of both alleles wherein 85.3% of these progeny were DsRed+ and yet a higher percentage (93.6%) were N^{Ax16} (FIG. 1E). Since loss-of-function null N⁻ alleles are non-viable in males, which carry only a single X chromosome, F2 ♂ were either N^{Ax16} or N⁺. Evidence that such lethal N⁻ alleles were indeed being generated as a result of imprecise DNA repair mediated by the non-homologous end joining (NHEJ) pathway is provided below. Additionally, consistent with the observation that nearly all F2 ♂ displayed a yellow⁻ mutant phenotype, sequence analysis of individuals from non-DsRed lines revealed that they all carried NHEJ-induced loss-of function mutations (FIG. 1D, FIG. 6).

Specifically, FIG. 6 shows a sequence analysis of Cas9-induced yellow-mutations on receiver chromosomes. DNA was isolated from y- DsRed-isogenic lines described in Table 2 and sequenced for NHEJ-induced mutations at the gRNA-y1 cleavage site. DNA sequences of several such alleles are presented, with in-frame y⁻ alleles highlighted.

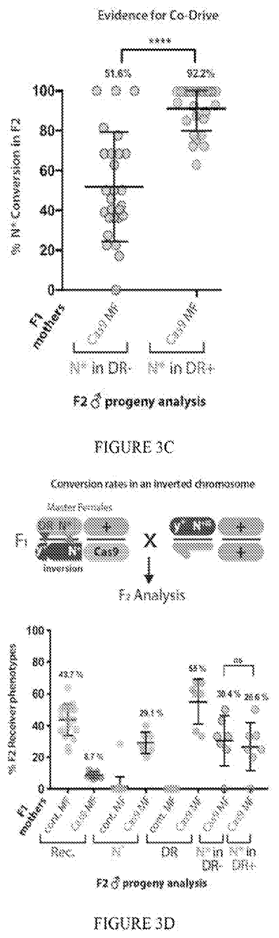

Super-Mendelian inheritance of the DsRed marked y^{<ccN>} element in F2 ♀ progeny (72.4%) was observed. However, Notch-related phenotypes can not be scored with certainty due to a high degree of mosaicism in which wings often displayed a mixture of wild-type, gain-, and loss-of-function phenotypes (FIG. 7). Such somatic mosaicism most likely results from the maternal perdurance of Cas9-gRNA complexes deposited into the egg, even in animals that did not inherit the Cas9-GFP transgene^{3,5,12,13}.

Specifically, FIG. 7 Shows mosaic phenotypes of F2 female progeny of master females. Examples of mosaic wing phenotypes observed in female F2 progeny of master females are depicted in FIG. 1D. Note the combination of phenotypes typical of Notch loss-of-function alleles consisting of wing margin notches and thickened veins (black arrows) and gain-of-function Abruptex alleles typified by distal vein truncations or gaps, which are particularly prominent in the L4 and L5 veins (gray arrows).

In order to circumvent this difficulty in scoring Notch phenotypes in F2 ♀, 104 individual lines derived from single F2 females (selected for absence of the Cas9-GFP transgene) were established, thus permitting unambiguous scoring of N phenotypes in subsequent generations (FIG. 1D). DNA was prepared from each isogenic line and PCR products from the Notch locus were sequenced to determine what alterations, if any, were present at the Notch-gRNA cleavage site (Table 2 and Table 3). DNA sequence polymorphisms located in an intron adjacent to the N^{Ax16} mutation (6.5 Kb upstream of the N^{Ax16} mutation) were identified that unambiguously distinguished the donor y^{<ccN>} N^{Ax16} bearing chromosome from the recipient chromosome (FIG. 8A). Results of the phenotypic and molecular analysis of the individual F2 ♀ lines are summarized in FIG. 1D (see Table 2 and Table 3 for extensive analysis and sequence data). Overall, these findings parallel those for F2 ♂ because super-Mendelian transmission of the DsRed marked y^{<ccN>} element (75%) and yet greater inheritance of the N^{Ax16} allele (87.5%) among the female founder lines was observed (FIG. 1D). Also, no N⁻ alleles were recovered, which in this case was surprising as such loss-of-function alleles generated by NHEJ would be expected to be viable in a heterozygous condition (see explanation below).

An important feature of the analysis of gRNA-induced events in the individual F2 ♀ lines was the ability to assign specific copying or non-copying events at the Notch locus to donor versus receiver chromosomes. In order to achieve the same end while analyzing larger numbers of progeny, the donor chromosome was marked with the tightly linked white-apricot (w³) allele (0.5 centimorgans from Notch, FIG. 8B), which causes an orange eye phenotype (i.e., y^{<ccN>} w³ N^{Ax16}). F1 master females (y^{<ccN>} w³ N^{Ax16}/y⁺ w⁻ N⁺; Cas9/+) were crossed to w⁻ males (FIGS. 8A-8B), and the resulting F2 individuals could be scored with ˜99.5% precision for inheritance of donor (w³) versus receiver (w⁻) chromosomes.

Specifically, FIGS. 8A-8B show molecular and genetic markers for scoring donor versus receiver chromosomes. FIG. 8A shows molecular markers; Notch intron DNA sequence polymorphisms used to score donor (A→T) versus receiver (6 nt deletion) chromosomes. FIG. 8B shows genetic markers: Alleles of the white locus, which is closely linked to Notch (0.5 centimorgans) were used to distinguish individuals carrying donor (w²=orange eyes) from receiver (w⁻=white eyes) chromosomes.

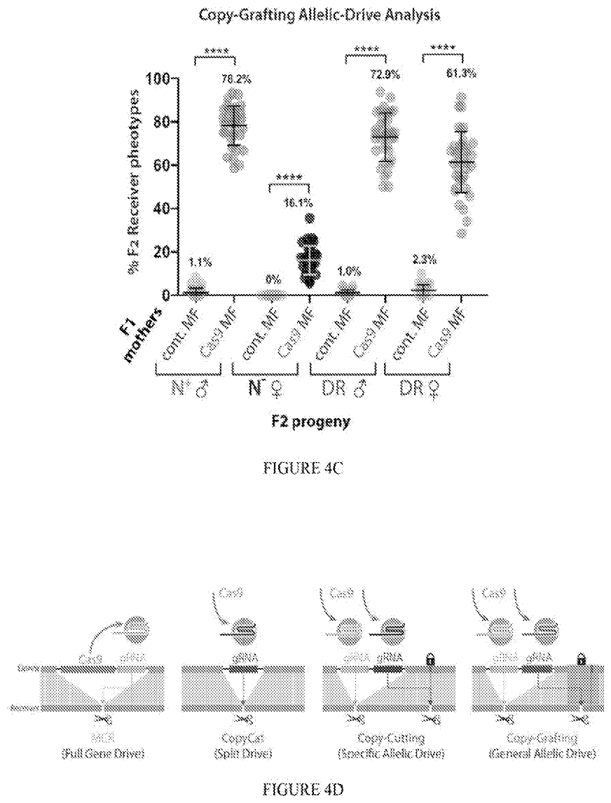

Compiled results from ˜20 such crosses conducted in parallel (FIG. 1F) reveal the same overall trend regarding the conversion frequencies observed in the F2 ♀ lines (FIG. 1D), with allelic conversion rates of N⁺ to N^{Ax16} averaging to 79.5%, a rate approximately a third higher than for copying of the y^{<ccN>} CopyCat element (˜60%). This is particularly notable given that gRNA-induced cleavage events at the yellow locus (100% in this invention—FIG. 6—) were consistently greater than at the Notch site, where 8-10% of progeny typically retained an unaltered wild-type target sequence (FIG. 1D, Table 2, and FIG. 9B).

Specifically, FIGS. 9A-9C show sequence analysis of Cas9-induced Notch mutations and allelic variants. FIG. 9A shows loss of function Notch alleles, Highlighted sequences are in-frame indels. FIG. 9B shows Notch alleles inherited from master females with wild-type function. Note that out of 35 such alleles sequenced (derived from isogenic females plus additional independently generated wild-type alleles): 28 matched that of the original receiver chromosome, 6 independent mutations were recovered in with a C→A mutation at the −4 position relative to the PAM sequence (e.g., immediately 5′ to the gRNA cut site (indicated by dashed vertical line), and one compound mutant with a C→A mutation at the −4 position and CA→GC alterations at the −2, −3 positions. FIG. 9C shows NHEJ-induced Abruptex alleles. The TCC deletion at position −2 −1 +1 was recovered 6 independent times, while two other Ax mutations were recovered only once.

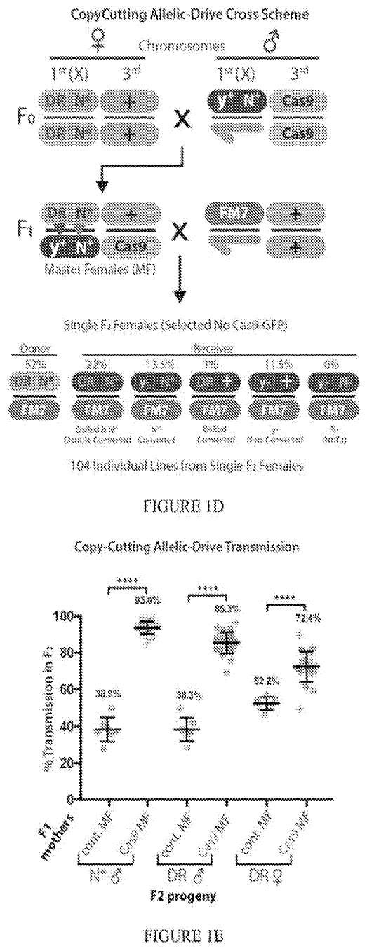

The relative proportions of receiver versus donor chromosomes in F2 progeny of F1 master females were examined and a ˜2-fold overabundance of donor chromosomes in males and a more modest, but highly statistically significant (p<0.0001 unpaired parametric t-test analysis), parallel bias in females (FIG. 2A, dark gray circles) was observed. In contrast, among control crosses (for which no source of Cas9 was introduced), a greater proportion of male progeny inherited the wild-type (receiver) chromosome than the N^{Ax16} donor chromosome, presumably reflecting a fitness cost associated with the N^{Ax16} allele (FIG. 2A, light grey circles).

Specifically, FIGS. 2A-2E show drive-induced lethal events on the receiver chromosome. FIG. 2A shows the frequency of inheriting the receiver chromosome from F1 master females (dark gray circles) compared to control F1 females lacking Cas9 (light grey circles) in males versus females. FIG. 2B shows overnight embryo collections stained with an antibody against the pan-neural Elav protein. Embryos from wild type females display normal central (CNS) and peripheral (PNS) nervous systems. Embryos derived from F1 master females reveal a classic neurogenic phenotype in which nearly all cells derived from the ventral neuroectoderm develop as neurons. N⁻ control embryos collected from N^{55e11/+} mothers. FIG. 2C shows frequencies of embryos displaying neurogenic phenotypes in collections derived from control w− mothers or from F1 master females crossed either to wild-type (WT; actually w⁻) ♂ or to N^{Ax16} ♂. FIG. 2D shows on the top panel: Sequence of a cleavage-insensitive N⁺ allele (N^{+IS}). Note the nucleotide change from C→A at position −4 relative to the PAM sequence for the N^{+IS} allele (which results in the phenotypically silent S→Y amino acid substitution. Middle panel: Crossing scheme in which F1 master females are mated to males carrying a wild-type N^{+IS} allele. Other diagram elements are the same as in FIG. 1D. Lower panel: percentage of adult female progeny displaying dominant N^{-/+} heterozygous wing phenotype consisting of wing margin notches and thickened veins (see FIG. 1C). The presence of the N^{-/+} phenotypic class was strictly Cas9 dependent. FIG. 2E shows on the left panel: crossing scheme for testing inheritance potential of an NHEJ-induced N⁻ allele by progeny of of y^{<ccN>} N⁻/Balancer females crossed to males carrying a vasa-Cas9 source on the third chromosome. Right panel: experimental results for three different NHEJ-induced N⁻ alleles (N⁻¹⁷, N⁻²⁰, and N⁻²¹) revealing that zero progeny were recovered from ccN N⁻ females carrying any of these three N⁻ alleles in the presence of zygotically provided Cas9.

One explanation for the pronounced Cas9-induced reciprocal inheritance bias of the N^{Ax16} donor allele, is that a fraction of gRNA-N+ induced cleavage events at the Notch locus on the receiver chromosome may result in NHEJ-induced N⁻ loss-of-function alleles. In males, such alleles would be hemizygous lethal resulting in strong embryonic neurogenic phenotypes causing much of the ventral epidermis to differentiate inappropriately as nervous system¹⁴. To test this, embryos from F1 master females (y^{>ccN>} N^{AX16}/++; Cas9/+ ♀) were collected and crossed to wild-type ♂. An abundant class of mutants (˜20%) with strong neurogenic phenotypes (FIGS. 2B-2C) was observed. As expected, neurogenic mutant embryos were absent from control crosses (i.e., from F1 y^{>ccN>} N^{Ax16}/++ ♀, FIG. 2C).

### Dominant Elimination of Lethal NHEJ Alleles

While the above results provide strong evidence for Cas9-dependent generation of N⁻ alleles in the progeny of F1 master females, they do not readily account for the failure to recover any heterozygous N⁻ alleles among the isogenic F2 ♀ lines. One possibility is that if F2 ♀ progeny derived from F1 master females inherit a N⁻ allele from their mothers, the “wild-type” paternal allele might be acted on in a mosaic fashion by maternally perduring Cas9-gRNA complexes^{3,5,12,13}. Such a maternal effect could result in a large enough proportion of somatic cells having two mutant copies of the Notch gene to preclude viability in trans-heterozygotes. This hypothetical mechanism, hereinafter referred to as lethal mosaicism, is consistent with the high frequency of mosaic wing phenotypes in F2 ♀ females as observed in this invention (FIG. 7). One prediction of the lethal mosaic hypothesis is that if F1 master females were crossed to males carrying the non-cleavable N^{Ax16} allele instead of wild-type males, the observed fraction of N⁻ F2 embryos should decrease significantly as a result of rescued lethality in females. This was indeed the case (FIG. 2C).

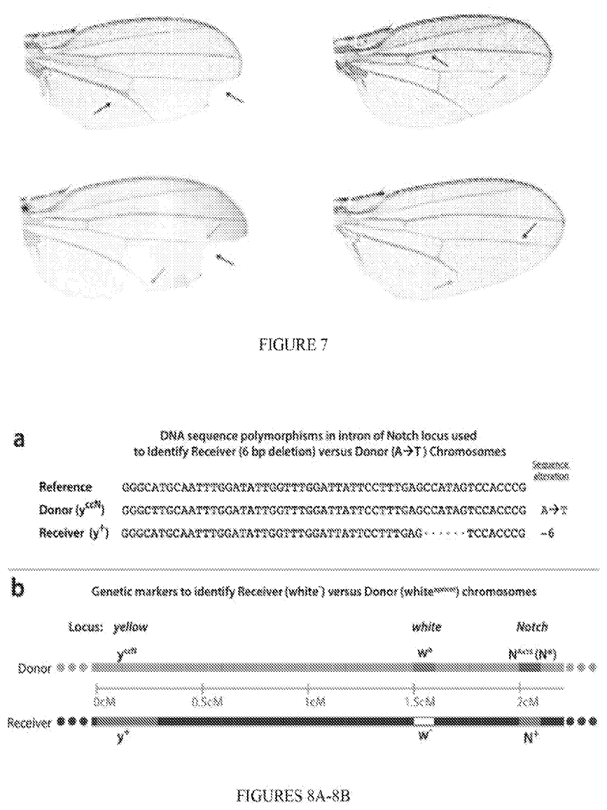

Another avenue for testing the lethal mosaicism hypothesis was provided by sequencing single F2 ♀ lines that inherited phenotypically wild-type N⁺ alleles (FIGS. 9B, 10). This analysis revealed that while most of these “wild-type” N⁺ alleles matched the original native allele, one had point mutations in the gRNA core sequence (i.e., between 1-4 nucleotides from the PAM site). Such mutations would be predicted to result in cleavage resistant or “insensitive” (N^{+IS}) alleles (see FIG. 2D for sequence of specific N^{+IS} allele used below). As in the case of crossing master F1 females to N^{Ax16} ♂, it was expected that crossing them to N^{+IS} ♂ (FIG. 2D) would also protect heterozygous female F2 progeny from lethal mosaicism since such individuals would carry one cleavage resistant functional N⁺ allele. The advantage of using N^{+IS} over N^{Ax16} fathers for such experiments is that N⁺/N^{+IS} ♀ heterozygotes are fully viable, whereas N⁻/N^{Ax16} ♀ exhibit significantly reduced viability. As predicted by the lethal mosaic hypothesis, a significant percentage (average=22%; p<0.0001 unpaired parametric t-test analysis) of F2 females derived from crosses of F1 master females to N^{+IS} ♂ displayed a typical heterozygous N^{-/+} wing phenotype in a Cas9-dependent fashion (FIG. 2D). Sequencing of several of such N⁻ alleles from individual F2 females revealed an array of DNA alterations centered at the cut site consisting of frameshifts, amino acid substitutions, and deletions (FIG. 9A, 10). In addition, consistent with the embryo studies described above, from which it was inferred that F2 females can be rescued from mosaic lethality by carrying a cleavage-insensitive Notch allele (FIG. 2C), a 20% excess of females to males (p<0.0001 unpaired parametric t-test analysis) among adult F2 progeny derived from crosses of master females to N^{+IS} ♂ was observed, which was not evident in parallel crosses to wild-type ♂ bearing the sensitive N⁺ allele (FIG. 11).

Specifically, FIG. 11 shows rescuing lethal mosaicism in female F2 progeny skews the fraction of receiver chromosomes recovered in males. Male and female F2 recipients of the receiver chromosome were recovered at approximately equal frequencies when fathers provided a N⁺ sensitive allele (N^{+S}). In contrast, when fathers provided a N⁺ insensitive allele (N^{+IS}), which protects female offspring from lethal mosaicism, a strong skewing against males was observed among F2 progeny inheriting a receiver chromosome. This is additional evidence for lethal mosaicism in F2 females, in which the paternal N allele determines lethality (N^{+S}), or survival (N^{+IS}) of the heterozygous female offspring.

An additional prediction of the lethal-mosaic hypothesis is that progeny inheriting the ccN drive element and any NHEJ-induced N⁻ allele from their mothers should be inviable if they also carried a zygotic source of vasa-Cas9. This was tested by establishing three different lines in which NHEJ induced N⁻ alleles (N⁻¹⁷, N⁻²⁰, and N⁻²¹—FIGS. 9A-9C) were associated with the ccN element. When such F0 females (y^{<ccN>} N⁻/Balancer) were crossed to homozygous Cas9 males (FIG. 2E), the only viable female F1 adult progeny recovered carried the N⁺ balancer (BAL) chromosome (FIG. 2E). The absence of emerging N⁻ F1 females in the Cas9 crosses contrasts with the expected 50% Mendelian transmission rate observed in non Cas9 control crosses. These experiments demonstrate that NHEJ-induced N⁻ alleles, such as those created in the germline of master females, cannot be transmitted to the next generation in the presence of Cas9 since they produce fully penetrant dominant lethality in heterozygous animals carrying the ccN element.

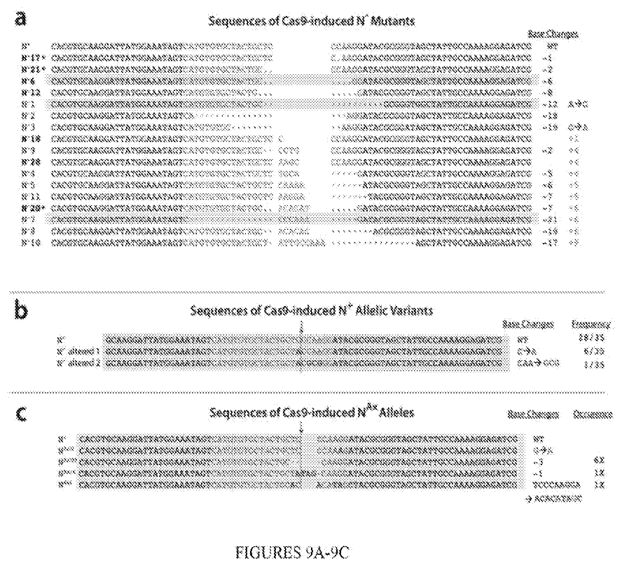

As a yet more stringent test of the lethal mosaic hypothesis, both males and females carrying the ccN element combined with a wild-type cleavage sensitive N⁺ allele (N^{+S}) were separately crossed to flies carrying the vasa-Cas9 transgene. Again, no surviving F1 progeny inherited the y^{<ccN>} allele (FIGS. 12A-12B).

Specifically, FIGS. 12A-12B show 100% lethality induced by the ccN CopyCat element in presence of two sensitive Notch wild-type alleles and Cas9. In FIG. 12A: Left panel: males carrying the ccN element and a wild-type Notch allele were crossed to females carrying two wild-type Notch (N^{+S}) alleles and a homozygous vasa-Cas9 transgene on the third chromosome. Right panel: no viable female progeny were recovered from this cross (circles with red outlines), while male progeny that do not inherit the ccN element were recovered in similar numbers as in control crosses (circles with black outlines). Many of the female progeny died at early pupal stages. Lower left panel: a late stage control pupa of the genotype N^{+S}/N^{+S}; Cas9/+ (black arrows labeled “e” indicate the developing eye primordium and white arrows labeled w, point to the wing primordium): Bottom left panel: a dead early stage female pupa of the genotype; y^{<ccN>} N^{+S}/N^{+S}; Cas9/+ (no eye or wing primordia are visible, but necrotic bodies are apparent, white arrow). In FIG. 12B: Females carrying the ccN element and two wild-type Notch alleles were crossed to males carrying a wild-type Notch allele and also homozygous for the vasa-Cas9 transgene. No viable progeny were recovered from this cross since they all carried the ccN element, a wild-type Notch allele, and Cas9.

Lethal mosaicism is a highly potent process that eliminates all progeny carrying the y^{<ccN>} allele, a hemizygous or homozygous cleavage-sensitive Notch allele, and a vasa-Cas9 source (either maternally or paternally provided). Moreover, progeny maintaining an association between the y^{<ccN>} and N^{Ax} alleles (or the very rarely generated N^{+IS} cleavage insensitive alleles) can survive in the presence of Cas9.

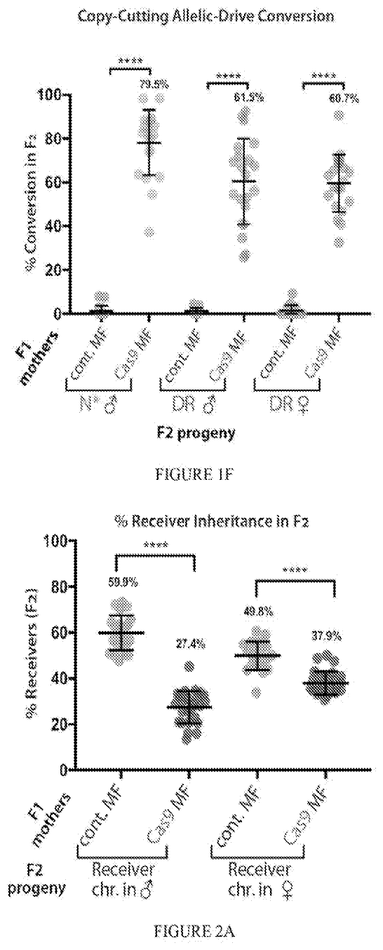

The observation of pervasive somatic and lethal mosaicism in crosses of F1 master females to wild-type males raised the possibility that maternally inherited Cas9-gRNA complexes in F2 ♀ might also persist and act in the germline to generate some degree of gene-drive or allelic-drive in the F3 generation, even in animals that did not inherit the Cas9 transgene. This was tested by crossing F2 y^{<ccN>} N^{Ax16}/++; +/+ (non-Cas9) ♀ to N^{+IS} ♂ (FIG. 3A).

Specifically, FIGS. 3A-3D show shadow drive and co-drive analyses. FIG. 3A shows three generation crossing scheme for analyzing shadow drive in the F3 generation. F2 ♀ derived from 1 master females were crossed to N^{+IS} ♂, and F3 progeny were scored for percentage conversion of F2 receiver chromosomes, and aeneration of N⁻ alleles. FIG. 3B shows a percent of F3 progeny demonstrating features of drive including (from left to right): percent of receiver chromosomes converted to N^{Ax16} allele (N⁺, blue circles; presence or absence of Cas9 refers to F1 generation); percent of heterozygous N^{-/+} females (N⁻, black circles—clustered around 12%); and percent of individuals having copied DsRed marked y^{<ccN>} element to receiver chromosomes in either males or females (red circles-clustered around 29.3% and 31.8%). The low percentages shown in control crosses (grey circles), in which F1 females lacked source of Cas9, presumably reflect the low rates of recombination between w² and N^{Ax16} (0.5 cm) or the y^{<ccN>} element (1.5 cm). FIG. 3C shows evidence for co-drive of N^{Ax16} (N⁺) allele with DsRed (DR+) marked element among individuals inheriting receiver chromosomes. FIG. 3D shows chromosome pairing is required for efficient allelic-drive and co-drive. Top panel: Genetic crossing scheme depicting allelic-drive to a balancer chromosome (Basc) that sustains˜normal chromosome pairing for the yellow locus but not for Notch. Lower panel: Experimental results showing˜normal levels of DsRed conversion of the to Basc chromosome, but significantly reduced copying (˜⅓) of the N^{Ax16} allele. In addition, co-drive of the DsRed and N^{Ax16} alleles was abolished by the Basc inversion.

F3 progeny from this cross did indeed manifest a substantial degree of perduring germline Cas9/gRNA activity as indicated by several measures, including: 1) copying of the DsRed-labeled y^{<ccN>} element onto the y+ N+ receiver chromosome (29.3% and 31.8% conversion of the receiver chromosome in males and females, respectively), 2) copying of the N^{Ax16} allele (27% conversion of the receiver chromosome in males), 3) recovery of N⁻/N⁺ ♀ (12%), and 4) Cas9-dependent depletion of receiver chromosomes in males (40.8% compared to 51.1% in control animals). Each of these measures of residual drive observed in the F3 generation (FIG. 3B), hereinafter referred to as shadow drive, was considerable, amounting to roughly half of that observed in the prior F2 generation.

### Co-Transmission of a Protected Allele and Drive Element

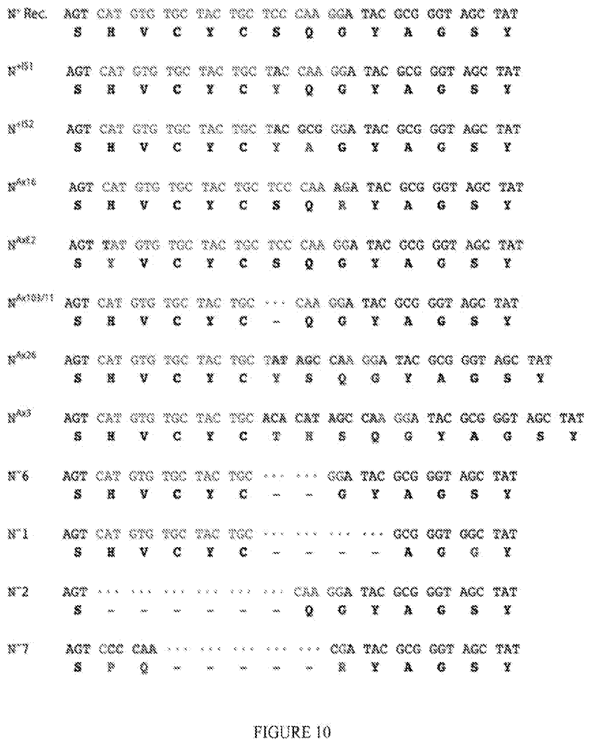

One surprise emerging from analysis of the copying efficiency of the y^{<ccN>} CopyCat element versus the N^{Ax16} allele, which was also a striking trend in the 104 single female lines (FIG. 1D), was an unexpected high positive correlation between these two conversion events. In nearly all cases where the CopyCat element was copied to a receiver chromosome, so too was the N^{Ax16} allele. This co-drive phenomenon can be most readily appreciated by considering the fraction of N^{Ax16} conversion events among DsRed+ (y^{<ccN>}) receiver chromosomes (92.2%), which is a frequency nearly double that of N^{Ax16} conversion among receiving chromosomes that failed to copy the y^{<ccN>} element (51.6%) (FIG. 3C). Similarly, among the single F2 female lines, 96% of the receiver chromosomes carrying the DsRed+ y^{<ccN>} element also copied the N^{Ax16} allele, in contrast to only 38% of progeny having copied the N^{Ax16} allele to a receiver chromosomes in the absence of y^{<ccN>} copying (FIG. 1D). Whether this co-drive phenomenon depended on chromosome pairing by placing the y^{ccN>} N^{Ax16} chromosome in-trans to a multiply inverted balancer X chromosome (Basc) was examined. This chromosomal arrangement resulted in a loss of co-drive as well as a marked reduction in the frequency of N^{Ax16} copying (FIG. 3D). It is notable that the frequency of DsRed copying in these crosses was only slightly reduced relative to that observed with a wild-type receiver chromosome, consistent with the tip of the Basc chromosome, which includes the yellow locus, being co-linear with wild-type chromosomes (note: the Basc IB3 inversion breakpoint is located to between y and N). Whether co-drive of the N^{Ax16} allele depended on active copying at the yellow locus by placing the y^{<ccN>} N^{Ax16} chromosome in-trans to a receiver chromosome carrying an NHEJ induced point mutation at the y-gRNA cleavage site was also tested. In this “single cut” configuration, reduced transmission of the N^{Ax16} allele relative to that in control crosses using a wild-type (y⁺N⁺) receiver chromosome was observed (FIGS. 13A-13B). This latter experiment indicates that copying the y^{<ccN>} element increases allelic-drive at the N locus.

Specifically, FIGS. 13A-13B show that allelic conversion at the Notch locus is reduced when copying of the ccN element at yellow is blocked. FIG. 13A is a crossing scheme. Top (double conversion—see results presented in FIG. 1F): control copying of both the DsRed (DR) marked y^{<ccN>} and Ax (N*) alleles, which leads to co-drive and high level copying of the Ax allele (97.3% in this experiment—panel b); Bottom (single conversion—see results in panel b): copying of the DsRed marked ccN element is blocked by a cleavage insensitive NHEJ allele at the y-gRNA cut site leading to no drive of that element and reduced drive of the Ax allele (panel b). FIG. 13B shows conversion percentages for double and single conversion schemes showing complete inhibition of ccN element copying by the y− NHEJ allele at the y-gRNA cut site and reduced (86.4%) copying of the Ax allele relative to that in control double conversion crosses (97.3%). Note that the observed levels of conversion for both the ccN element and Ax16 allele in control double conversion crosses were higher than in previous experiments, which may reflect the fact that an additional generation was required to generate master females for these crosses, which perhaps reduced the effect of suppressor mutations limiting Cas9 expression/activity. Such suppressors often accumulate in stocks over time.

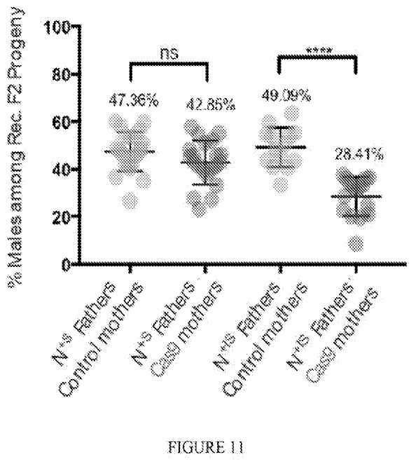

Finally, the impact of cis- versus trans-configurations for the y^{<ccN>} and N^{Ax16} alleles was examined by generating master females of the genotype y^{<ccN>} N^{+S}/y⁺ N^{Ax16}; Cas9/+ (FIGS. 14A-14B) and efficient drive of both the DsRed marked y^{<ccN>} element (76% conversion) and the N^{Ax16} allele (93% conversion) was observed indicating that both cis- and trans-configurations of the two elements sustain potent drive.

Specifically, FIGS. 14A-14B show efficient allelic drive is observed when the N^{Ax16} allele is in a trans-configuration relative to the y^{<ccN>} allele. FIG. 14A shows crossing schemes depicting the standard cis-conversion drive (top—see results presented in FIG. 1F) and trans-configuration drive (panel b) wherein the DsRed marked ccN element is linked to the w⁻ and N^{+S} alleles, and the N^{Ax16} allele is on a y+ w² chromosome. FIG. 14B shows percentages of bi-directional conversion rates (e.g., DsRed w² or Ax w⁻). Both trans-copying events occur with high efficiency.

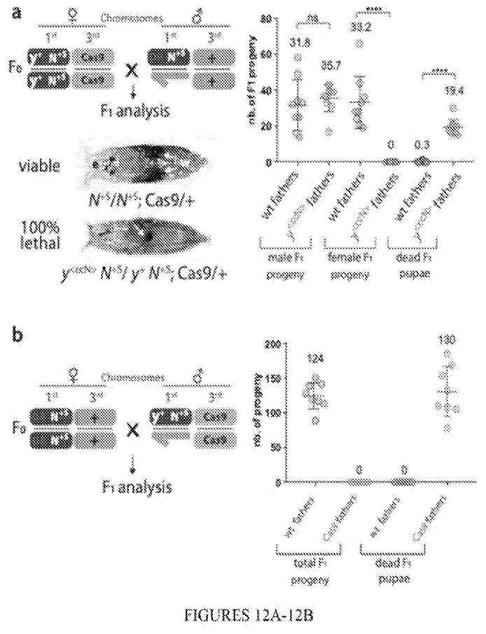

While copying of the Ax16 allele from the donor to the receiver chromosome was the overwhelmingly prevalent event in the above allelic drive experiments, rare in-frame NHEJ-induced indels induced at the gRNA-N+ cut-site that generated de-novo Abruptex alleles were also identified (FIGS. 9C, 10). Whether it was possible to produce allelic drive with one such de-novo allele (Ax103) by recombining N^{Ax103} with the y^{<ccN>} element was tested. It performed equivalently to the N^{Ax16} allele in driving conversion of a wild-type N⁺ allele to an Abruptex phenotype (FIG. 15). This finding demonstrates that allelic variants mutating residues at the Cas9 cleavage site are equally well-suited as those disrupting a PAM site for sustaining efficient allelic drive. Specifically, FIG. 15 shows efficient allelic drive of the de novo cleavage site N^{Ax103} allele. The de novo N^{Ax103} allele (recovered independently 4 times) is an in-frame deletion of 3 nucleotides spanning positions −3 to −5 relative to the PAM sequence that eliminates a serine codon present in the wild-type Notch sequence. Master females carrying the N^{Ax103} allele transmitted the DsRed and Ax phenotype and converted the receiver (w⁻) target chromosome to Ax at frequencies similar to those observed with the PAM disrupting N^{Ax16} allele. These results suggest that copy-cutting is also efficient for various alleles that disrupt residues critical for gRNA target site recognition.

Copy grafting: is a broadly applicable allelic drive strategy. The experiments described above demonstrate highly efficient allelic-drive of the N^{Ax16} allele via copy-cutting mechanism, exceeding by nearly a third that observed for the y^{<ccN>} gene-drive CopyCat element. While these results are encouraging, the obvious limitation of such a strategy lies in the requirement for a gRNA to selectively cut the targeted undesired allele. This constraint requires that the preferred allele either lacks a PAM site or differs from the targeted allele in core gRNA sequences (˜1-5 nucleotides from the PAM site), which would occur in only a fraction of cases (˜60% of single nucleotide polymorphisms if GG di-nucleotides occur at a frequency of 1/16 and are randomly distributed). A more general allelic-drive method was also developed in this invention by making use of the fact that homology directed repair (HDR) is often accompanied by local gene conversion events spanning as much as several hundred nucleotides from the double stranded cleavage site¹⁵. This local repair phenomenon has been well documented in Drosophila¹⁶⁻¹⁸, and may reflect the range of 3′ resection during the DNA repair process¹⁶.

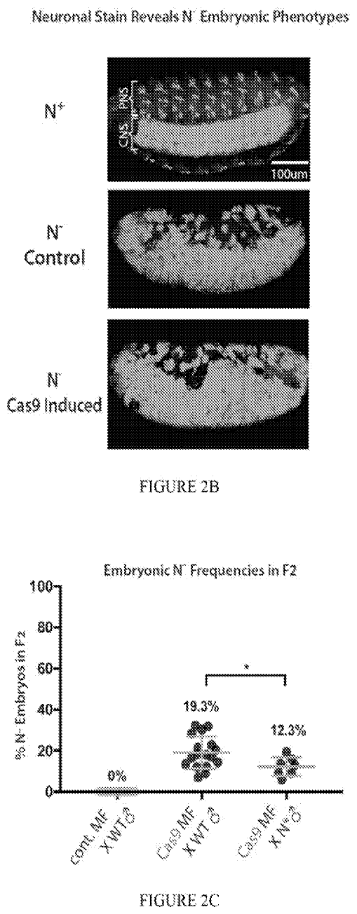

With the above motivating concept in mind, and the rich array of genetic variants available in Drosophila, as well as those generated in this study, a reverse-drive scenario wherein the wild-type N^{+IS} allele should be preferentially inherited over a different cleavage-sensitive, Abruptex allele (AxE2) was conceived. The N^{AxE2} allele results from a C→T substitution located 21 bp upstream of the cleavage resistant N^{Ax16} G→A alteration (FIG. 4A). The N^{AxE2} allele, however, should be sensitive to cleavage by the gRNA-N+ carried on the y^{<ccN>} CopyCat element since it lies at a sufficient distance from the gRNA cut site (17 bp, FIG. 4A, N^{AxE2} indicated as N*^{S} in FIG. 4B).

Specifically, FIGS. 4A-4D show an allelic drive mediated by “copy-grafting”. FIG. 4A shows DNA sequence at the gRNA-N+ cleavage site for the wild-type reference allele (N^{+S}), the cleavage-insensitive wild-type (N^{+IS}), and the cleavage-sensitive N^{AxE2} allele (N*^{S}), with the C→T substitution (see FIG. 10 for amino-acid substitutions). FIG. 4B shows a copy-grafting scheme in which F1 master females carrying a wild type cleavage insensitive wild-type Notch allele in trans to the sensitive N^{AxE2} allele (y^{<ccN>} w³ N^{+IS}/y⁺ w⁺ N^{AxE2}; Cas9/+ ♀) are crossed to N^{+IS} ♂. F2 progeny were then scored based on inheritance of the y^{<ccN>} w² N^{+IS} donor chromosome or the w⁺ receiver chromosome (dark gray) based on their eye color phenotype (orange for donor and red for receiver). Arrowheads indicate copying of the y^{<ccN>} element and the N^{+IS} allele respectively. FIG. 4C shows a percent of F2 progeny demonstrating features of drive including: percent of converted N^{+IS} receiver chromosomes (gray circles—clustered around 78.2%); heterozygous N^{-/+} females (black circles); and copying of the DsRed marked y^{<ccN>} element to receiver chromosome (gray circles—clustered around 72.9% and 61.3%). FIG. 4D shows a summary of different drive systems. MCR (full gene-drive) elements have both Cas9 and a gRNA inserted into the genome at the gRNA-directed cleavage site, CopyCat (split-drive) elements carry only the gRNA inserted at the gRNA-directed cleavage site, while a Cas9 is provided from a Mendelian transgene at a different genomic location. Copy-cutting (special allelic-drive) is mediated by two gRNAs. One gRNA (light gray) propagates the CopyCat element while the second gRNA creating allelic-drive (dark gray) cuts a non-preferred allele, but not the favored allele (lock icon). This drive element can be either an MCR (including Cas9) or a CopyCat element (Cas9 provided from a separate genomic location, as depicted in this figure). Copy-grafting (general allelic-drive) is mediated by a gRNA that cuts the non-preferred, but not the favored, chromosome near the desired allelic variant, resulting in conversion of a short region of the receiving chromosome (indicated by highlighting) with sequences from the donor chromosome (gray box) that encompass the favored allele (lock icon).

If the y^{<ccN>} CopyCat element were recombined with the wild-type N^{+IS} cleavage insensitive allele, which carries a single nucleotide change (C→A) at the −4 position (FIG. 4A), it might be possible to drive that wild-type N allele onto a receiving chromosome carrying the N^{AxE2} allele. This inverse-drive scheme depicted in FIG. 4B is referred to as copy-grafting.

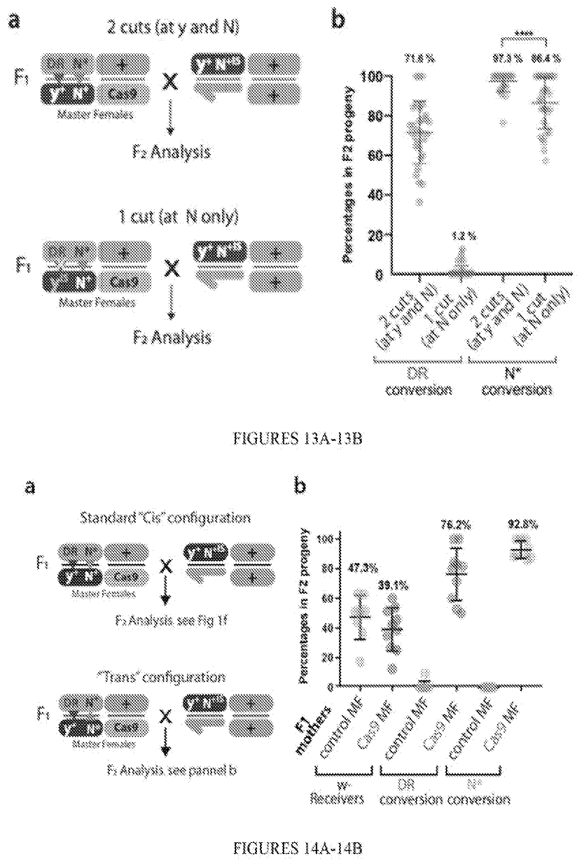

Results in FIG. 4C reveal that the efficiency of inverse allelic-drive via copy-grafting (78.2% conversion of receiver chromosomes in males) was comparable to that observed by copy-cutting (79.5% conversion—FIG. 1F). Also, as was observed for copy-cutting, copy-grafting resulted in a deficiency of receiver chromosomes in F2 ♂ progeny, in the generation of N⁻ alleles (˜16%; when F1 master females were crossed to N^{+IS} ♂ to protect the paternal chromosome from lethal mosaicism), and in strongly correlated co-drive between the y^{<ccN>} and N^{AxE2} alleles (FIG. 16).

Specifically, FIG. 16 shows copy-grafting sustains efficient co-drive. The frequency of allelic conversion to the N^{+IS} phenotype among receiver chromosomes (marked with the w⁺ allele) was tabulated among individuals in which the y^{<ccN>} CopyCat allele had also copied or not. As in the case of copy-cutting drive of the N^{Ax16} allele, a strong enrichment of N^{+IS} copying events was observed among those that had also copied the y^{<ccN>} allele relative to those that did not.

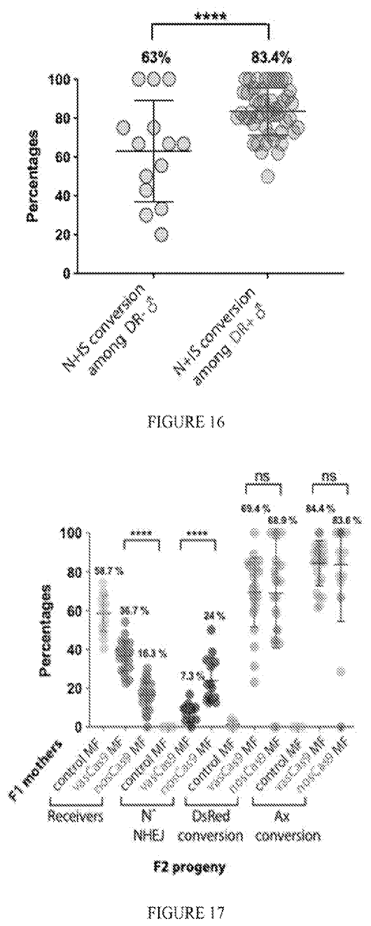

FIGS. 18A-18B show preliminary cage trial experiments for copy-grafting allelic-drive in a Cas9 recipient population. In FIG. 18A, the ccN drive element and favored non-cleavable allele N+IS were introduced through virgin master females (y<ccN> N+IS/+ N*S; Cas9/+) into a recipient population carrying a cleavage-sensitive Notch allele and homozygous for a vasa-Cas9 transgene (+N*S/+ N*S; Cas9/Cas9). Note that the Cas9 transgene carries a functional yellow transgene marker. In flies carrying the drive, this y+ marker is mutated to y− in a Cas9-dependent fashion, resulting in an initial fitness disadvantage relative to the y+ recipient population (y− males suffer a severe competitive mating disadvantage, a well-known phenomenon⁽³⁶⁾. This y−/y+ assortative mating barrier is likely to be responsible for the initial dip in N+IS allelic frequency observed in males. Starting allele frequencies: Master females represent 25% of the initial population, the initial N+IS allelic frequency is 16.6%, and the overall prevalence of the Cas9 transgene is 87.5%. Control cages include the same initial genotypes at identical proportions without introduction of Cas9. Four independent cages were set up for vasa-Cas9 and control populations. FIG. 18B shows in the left panel: N+IS allelic frequency measured in males over 5 generations. ˜150 males were scored for each cage at each generation. By generation 5, the N+IS allele reached a near-fixation level (95.2%) in Cas9 cages, while remaining at a stable ˜50% representation over three consecutive generations in control cages. Note that the initial N+IS allelic percentage in males is 0%, since all males in the F0 population are N*S. Right panel: Presence of the ccN drive element measured in each population (males and females combined) through DsRed marker scoring. ˜300 individuals were scored for each cage at each generation. DsRed frequencies are higher (77.6%) in Cas9 populations than in control populations (62%). This relatively modest difference may be caused by the accumulation of drive resistant y− NHEJ alleles in the Cas9 population and may in part reflect the assortative mating disadvantage suffered by the male progeny of the initial F1 master females. Despite this less efficient drive of the ccN element, the N+IS allele is driven to high prevalence in Cas9 populations. This cross scheme simulates an important potential application of gRNA-only “CHACR” elements that can be used to update gene-drive content⁽⁶⁾. In this scenario, a full gene-drive element has spread to near completion in a population. In that background of broad Cas9 prevalence, a CHACR is released to improve some performance aspect of the gene-drive. The CHACR is expected to spread geometrically into the population following the same logistic growth curve as a full-gene drive in a naïve population.

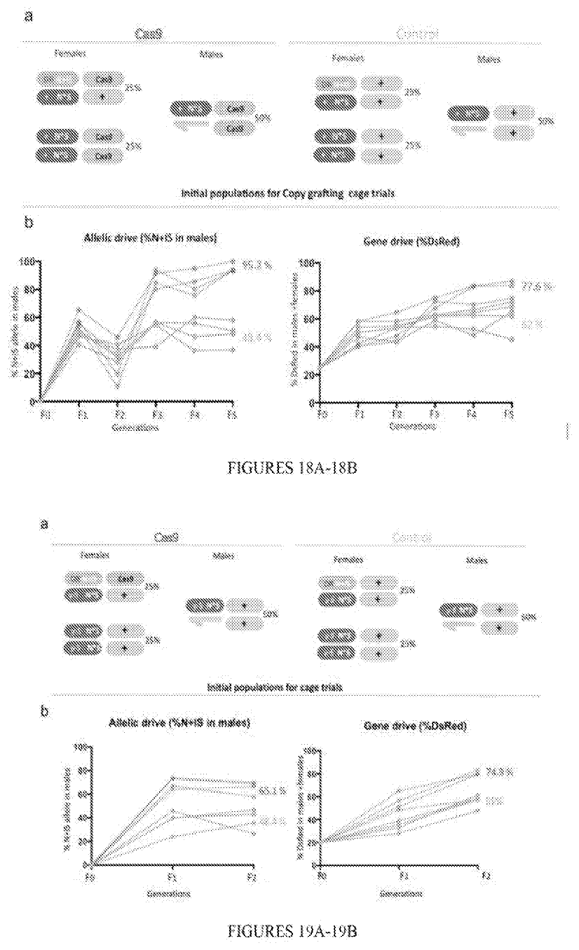

FIGS. 19A-19 show preliminary cage trial experiments for copy grafting in an allelic pump paradigm where a fixed percentage of Cas9+drive cassette (introduced at equal frequencies) are released into a naïve (non Cas9) recipient population. In FIG. 19A, the ccN drive element and a cleavage resistant Notch+ (N+IS) allele were introduced via virgin master females (y<ccN> N+IS/+ N*S; Cas9/+) into recipient population homozygous for the N*S allele (+N*S/+N*S; +/+). Starting allele frequencies: Master females represent 25% of the initial population, the initial frequency of the N+IS allele is 16.6%, and the seeding frequency of Cas9 is 12.5%. Control cages include the same initial genotypes at identical proportions without introduction of Cas9 from master females. Four cages were set up for Cas9 and control population. In FIG. 19B, in the left panel: N+IS allelic frequency measured in males for two generations. ˜150 males were scored for each cage at each generation. The N+IS allele has reached significantly higher frequency (65.1%) in Cas9 cages than in control cages (48.4%). As shown in FIGS. 18A-18B, the initial N+IS percentage in males is 0. Right panel: Presence of the ccN drive element measured in each population (males and females combined) through DsRed marker scoring. ˜300 individuals were scored for each cage at each generation. DsRed frequencies are significantly higher (74.9%) in Cas9 populations than in control populations (55%). This cross scheme simulates an allelic pump scenario⁽⁶⁾ in which a fixed percentage of individuals carrying a Cas9 transgene and a gRNA-only cassette are released into a wild-type population. Under these conditions the increase in gRNA-cassette frequency is additive at each generation (i.e., initially equals the percent of Cas9 prevalence in the population=12.5% in this experiment). This allelic pump system represents the weakest form of a Cas9 driven bipartite element.

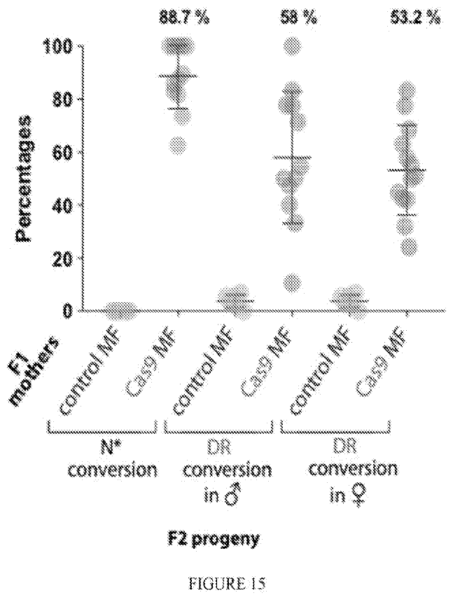

This invention has demonstrated the feasibility of two forms of allelic-drive: copy-cutting, which applies to cases in which a gRNA can be designed to selectively target a non-preferred allele; and copy-grafting, a more general strategy in which one associates a cleavage-resistant site in proximity to a favored allelic variant (FIG. 4D). Both allelic-drive systems are significantly more efficient than a gene-drive CopyCat element inserted into the yellow locus. An important mechanism contributing to the efficient allelic drive, which is of potential relevance to field applications, is the phenomena of lethal mosaicism, which dominantly eliminates all NHEJ-induced drive-resistant non-functional loss-of-function alleles of an essential gene such as Notch. Shadow-drive, in which perduring Cas9-gRNA complexes transmitted maternally for one generation in the absence of the Cas9 or gRNA transgenes, should also act as a genetic slingshot to extend gene or allelic-drives for one additional generation should they become separated from a Cas9 source. Interestingly, a strong correlation between copying events resulting in preferential transmission of both the gene-drive cassette and the preferred non-cleavable allele was demonstrated. This co-drive phenomenon depends on chromosome pairing, as it is not observed when the receiver chromosome carries an inversion affecting the targeted gene. Similar correlated genome editing events have been reported recently in other systems including in the germline¹⁹ and in somatic cells²⁰. With regard to copy-cutting, the recent development of Cas9 variants with broadened PAM specificity^{21,22} will substantially increase the fraction of alleles that can be driven by this method. Development of such systems should function with various sources Cas9, as allelic drive with a nanos-Cas9 was observed as efficiently as with the vasa-Cas9 source (FIG. 17). Altogether, the allelic drive strategies as demonstrated by this invention are broadly applicable to various gene targets in diverse experimental and agricultural contexts.

Specifically, FIG. 17 shows the nanos-Cas9 source sustains efficient allelic conversion, but also induces more male lethal NHEJ events than observed with the vasa-Cas9 source. From left to right: Percentages of F2 progeny inheriting a Receiver chromosome. Control crosses show a notable bias in favor of the w⁻ receiver chromosome allele (˜60% transmission) in male progeny, presumably due to a fitness cost associated with the N^{Ax16} allele present on the donor chromosome. Progeny from nanos-Cas9 master females show a much stronger reduction in receiver chromosome inheritance (16.3%) in comparison to what is observed with vasa-Cas9 (36.7%) master females (MF). This reduction is accompanied by an increase in the frequency of N⁻ NHEJ alleles recovered among F2 Receiver females from nanos-Cas9 master females. In contrast, the percent conversion of DsRed and the N^{Ax16} allele among receiver progenies from nanos-Cas9 versus vasa-Cas9 master females is not significantly different. Consistent with the hypothesis that the nanos-Cas9 source generates lethal Notch alleles more often than vasa-Cas9, a higher frequency of N⁻/N^{+IS} F2 females were recovered using nanos-Cas9 versus vasa-Cas9. Cumulatively these data suggest that the modest increase in apparent efficiency of the nanos-Cas9 versus vasa-Cas9 source in y^{<ccN>} and N^{Ax16} transmission reflects increased rates of generating lethal NHEJ (Notch-) alleles rather than an increase in the rate of copying (conversion).

One important category of potential applications of this invention is the aggregation of multiple favored naturally occurring allelic variants in plants or animals. In plants, allelic drive schemes can facilitate combining favorable traits to improve crop yields and resistance to environmental stresses, particularly in polyploid species (e.g., wheat or rye). Similarly in animal models, active genetics can accelerate the construction of complex genotypes in model organisms for biomedical and basic research. For example, one can envision crossing individual plants or animals bearing a favored allele to a strain carrying a Mendelian cassette with Cas9 and a gRNA that target the corresponding non-preferred alleles. The Cas9 bearing progeny also inheriting the first favored allele can then be crossed to a strain carrying the second favored allele and a guide RNA targeting the second unwanted allele, and so on until all favored alleles are gathered into a single strain. Finally, one can perform a final cross to segregate out the Cas9-gRNA cassettes. In polyploid crops such a strategy should permit assembly of several preferred alleles providing drought resistance²³, higher yields²⁴, optimal architectures²⁵, or more rapid growth²⁶ that would be long, difficult or impossible to assemble into a single strain by standard genetic crossing schemes.

Another important class of allelic-drive applications of this invention is to reverse pesticide resistance in pest species. Use of insecticides has repeatedly led to the emergence of specific insecticide-resistant alleles in insect disease vectors and crop pests. Many pesticides target essential components of the nervous system such as the Na⁺ channel or glutamate receptor²⁷. Allelic drive systems can help revert these populations back to their wild-type sensitive state, which would be aided by the reduced fitness of certain prominent insecticide resistant alleles in the absence of pesticide use²⁸⁻³⁰. Even modest reductions in the incidence of resistant alleles (e.g., to prevalence of <50%) can have major positive impacts on disease reduction strategies^{31,32}. Similarly, allelic drives can be used to favor genetic variants that prevent host species from serving as disease vectors or pests.

One potential concern in such allelic drive scenarios is whether NHEJ-induced cleavage resistant alleles can also be driven by the gRNA intended to drive the favored allele. This run-away NHEJ problem might arise if the primary drive cassette became separated from the preferred allele and instead became associated with an undesired NHEJ-induced allele. Several lines of evidence presented in this invention show that this scenario is unlikely so long as the gRNA sustaining allelic drive targets a critical region of an essential gene such as Notch (or the Na⁺ ion channel). Firstly, the strong co-drive greatly limits the number of events separating the favored allele from the drive element (only a few percent). Secondly, lethal mosaicism eliminated 100% of the progeny carrying three different NHEJ-induced N⁻ alleles and also killed all offspring carrying the gene-drive element and unprotected wild-type alleles. Finally, allelic-drives work very effectively in-trans as well as in-cis, so that should an uncoupling event occur, it would be rapidly reversed in most instances. Thus, all non-functional NHEJ alleles will be eliminated immediately as they are generated, and drive of the favored allele will persist either in-cis or in-trans. The only remaining concern is whether one might not occasionally create a functional non-cleavable version of the undesired allele that can then also be driven. While this is possible (e.g., rare N^{+IS} and novel Ax alleles were recovered), such events are very infrequent and should not drive any more efficiently than the overwhelmingly prevalent preferred allele. It is possible to further reduce the production of such rare events by using two gRNAs simultaneously, one directing copy-cutting and the other copy-grafting of the same preferred allele. Thus, it is practical to drive preferred alleles of essential genes efficiently into a population so long as they do not impart a significant fitness cost relative to the non-preferred allele.

Lethal mosaicism also has game-changing implications for developing new efficient gene-drive systems. A drive element targeting a critical site in a gene essential for viability or reproduction can also carry a functional recoded (and non-cleavable) portion of that same gene, thereby protecting progeny inheriting this element from lethal (or sterile) mosaicism. In contrast, non-functional NHEJ-induced mutations rendered dominant by lethal mosaicism are eliminated immediately, thereby “killing or sterilizing the mistakes” and providing a powerful solution to the frequently highlighted drive-resistance problem. These and other diverse applications of allelic-drive will greatly expand the impact of active genetics, accelerating progress in many areas of synthetic biology.

### Methods:

### Construction of ccN CopyCat Element

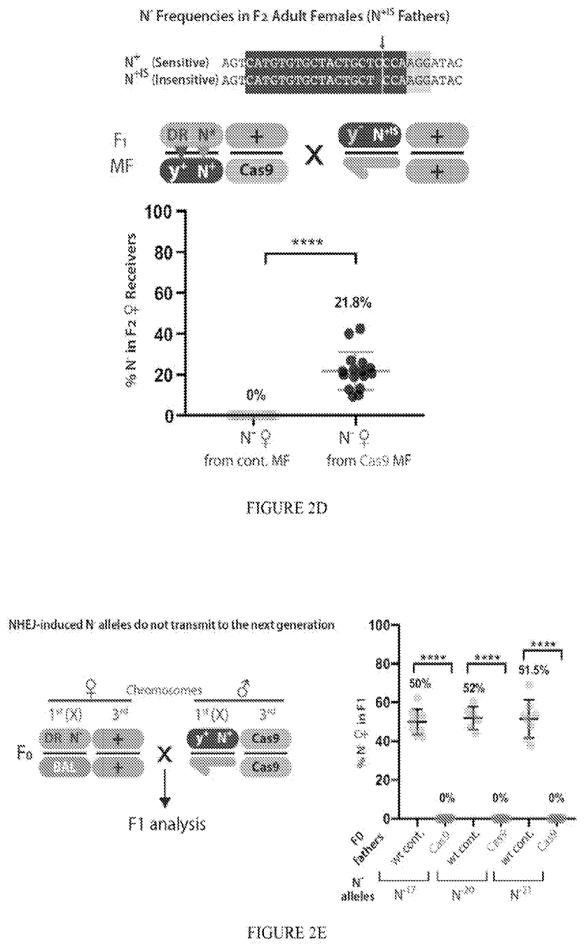

Cloning, of the ccN CopyCat plasmid followed the same strategy as described in Xu et al.⁷ using homology arms to the yellow locus abutting gRNA-y1 cleavage site and carrying gRNA-y1, gRNA-N+, and a 3XP3-DsRed eye marker as depicted in FIG. 5 (the full DNA sequence of this plasmid is provided in Table 1).

Specifically, FIG. 5 shows in the top panel: Circular map of ccN CopyCat element plasmid indicating the location of the homology arms for insertion into the yellow locus (yellow HA1, 2), a yellow guide RNA for insertion into and for copying at the yellow locus, a gRNA tor cutting the wild-type Notch locus but not Ax16 allele (gRNA-N+ in text), and a DsRed fluorescence marker. Bottom panel: Linear map showing integration of the ccN element into the yellow locus and the locations of primers used to PCR amplify fragments for sequencing. Insertion of the ccN element into its target site and its flanking genomic sequences were amplified using primers located beyond (˜300 bp) the HA1 and HA2 arms (pS1: CGACTCAGCTCACGTATTTCATACAG with pAS1: CTCCCCCTGAACCTGAAACATAAAATG, and pS2:CAGGAGCTCCAGCTTTTGTGCTAG with pAS2:GAGAATATACCCAAGTACCGTGTGTAG) and fully sequenced, which confirmed a perfect insertion of the element into the intended genomic target site. Sequences within the ccN element were identical to those in the vector shown in Table 1. The full nomenclature for the ccN CopyCat element according to previously established convention for active genetic elements^{1,2} is: y^{<CC|gRNA-y1, gRNA-N+|3XP3DsRed>}, hereinafter abbreviated in the text as y^{<ccN>} for convenience. The complete DNA sequence of the ccN element is provided in Table 1.

Following assembly of its components, the ccN CopyCat plasmid was transformed into ONE SHOT® TOP10 competent cells (Invitrogen #C4040) and purified using the Qiagen Plasmid Midi kit (#12191). An injection mix containing the ccN plasmid (final concentration: 250 ng/μl) was sent to Best Gene Inc. for injection into embryos collected from a w³ N^{Ax16} rb⁻ stock (which is resistant to the otherwise lethal mutagenesis of the Notch locus generated by Cas9/gRNA-N+) with a transient source of pHsp70-Cas9 (Addgene plasmid #45945). The w³ N^{Ax16} rb⁻ stock was kindly provided by Jim Posakony (UCSD). Male transformants carrying the ccN element were identified in F1 progeny by virtue of their yellow⁻ and DsRed fluorescent eye-marker phenotypes. This genomic insertional allele is referred to as: y^{<CC|gRNA-y1, gRNA-N+|3XP3DsRed>} in accordance with the previously established nomenclature convention^{6,7} (see FIG. 5 for details of the construct). For shorthand in the text, this allele is referred to as: y^{<ccN>}. The ccN plasmid construct was fully sequenced prior to injection as well as that of the ccN genomic insertion for several individual y^{<ccN>} transformant lines, which included PCR amplification and sequencing of endogenous sequences lying adjacent to those included as homology arm templates in the plasmid construct to verify accurate insertion of the ccN element into the intended site (Table 1).

Genomic DNA preparation: Genomic DNA from single adult flies were prepared according to protocols by Gloor et al.³³ Single flies were crushed in lysis buffer (10 mM Tris pH8.2, 1 mM EDTA, 25 mM NaCl, with 0.3 mg/ml proteinase K, added right before incubation), incubated at 37° C. for 30 min, and heated at 95° C. for 2 min. 100 μl of ddH₂O were added to each tube before storage at −20° C.

Drosophila genetics: Flies carrying the donor y^{<ccN>} w³ N^{Ax16} chromosome were identifiable through the visible w³ orange eye phenotype. y^{<ccN>} w² N^{Ax16}/FM7 females were crossed to Casty homozygous males (BL# 51324) to generate Fl master females as diagrammed in FIG. 1D. Crosses were performed at 25° C. on standard Drosophila food. The y⁻ w⁺ N^{AxE2} carrying a gRNA-N+ sensitive Abruptex allele was kindly provided by Spyros Artavanis-Tsakonas (Harvard University). A cleavage-insensitive N⁺ allele recovered among the 104 isogenic lines was recombined with y^{<ccN>} w² to generate the donor chromosome in FIG. 4B. For quantitative analyses of F2 progeny (or F3 progeny for the Shadow drive), 20-37 crosses consisting of 3 females mated to 3 males were analyzed for each experiment, yielding an average of ˜150 flies per cross. A total of 29,000 progeny were analyzed for this study.

Sequence analysis: To sequence mutations in the yellow locus, a ˜500 bp fragment was amplified by PCR (Q5 Hot Start High-Fidelity 2x Master Mix) with primers 417 (TTTAGTGCCTCAATAATAGTTTGGCCCTGC) and 356 (GGACATACCAAATATACCCTCC), then sequenced with primer 418 (GGAAGTTAATACCAGCGACATTGAAATCGC) at Genewiz. To identify donor vs receiver chromosomes, a fragment from Notch intron 5 was amplified with primers NintS3 (CTACGAGTGCAAGTGCCCCAAAG) and NintAS3 (CGCCCGGAACGTTGGAATGGAATG) and sequenced with NintS3bis (CAGTAGGAACCAGATTAATCGAGTT). For sequencing mutations in the the NAx region, primers NAxS (CCACGAGCAAAACAACGAGTACAC) and NAxAS2 (TTCGAATCACAATCCTGACCACTCAGC) were used to amplify a ˜1 Kb fragment, and sequenced using primer NAxS3 (GCATCAATGGCTACAACTGTAGC).

Active genetic safety measures: All crosses using active genetics were performed in accordance with an Institutional Biosafety Committee (IBC) approved protocol from UCSD in which full gene drive experiments are performed in a high security ACL2 barrier facility and split drive experiments are performed in an ACL1 insectary in plastic vials that are autoclaved prior to being discarded in accord with currently suggested guidelines for laboratory confinement of gene drive systems(^{34, 35}).

Wing dissection and mounting: Drosophila wings were dissected in Isopropanol and mounted in 100% Canada balsam.

Antibody Staining of Drosophila embryos: Fixation and antibody staining of embryos using a rat anti-Elav (DSHB #7E8A10, antibody dilution=1/20) was performed according to standard procedures. Samples were mounted in Slowfade diamond anti-fade mountant (Thermo Fisher Scientific #S36963) and imaged on a Leica SP5 confocal microscope. Each data point in FIG. 2C corresponds to the analysis of a group of 30-50 embryos on a slide. Embryos of stages 11-16 were scored for N⁺/N⁻ phenotypes using a Zeiss AXIO ZOOM V16 fluorescent microscope.

TABLE 1
Complete DNA sequence of ccN CopyCat element.

>pVG209_y1-Notch_in_y1-DSRED 2.dna (8907 bp)
ACCGCCTCTCCCCGCGCGTTGGCCGATTCATTAATGCAGCTGGCACGACAGGT
TTCCCGACTGGAAAGCGGGCAGTGAGC
GCAACGCAATTAATGTGAGTTAGCTCACTCATTAGGCACCCCAGGCTTTACAC
TTTATGCTTCCGGCTCGTATGTTGTGT
GGAATTGTGAGCGGATAACAATTTCACACAGGAAACAGCTATGACCATGATT
ACGCCAAGCTATTTAGGTGACACTATAG
AATACTCAAGCTATGCATCAAGCTTGGTACCGAGCTCGGATCCACTAGTAACG
GCCGCCAGTGTGCTGGAATTCGCCCTT
ctggagaactacattgcctgaattggcgggcaaataagtgcgacttggaggaggcggcgaggaagcccagctggtctgagg
tttctgtggcaagacaggacgatattgtttcgtatataacggtggacccattggcaaaacggcttgttttggtattgaaa
cggtatttggcggcccataggcgttattcctcaaatcacacacagtattctcaattaaagtggccaagggagccgtgtaa
attcggaaattagtatctgaataatccaagtcagacagcaagaaaacgggcatcctatcggatagaacccaaacgttttt
gttctcatcaattttcacatcggccggaaaaactaagccaacgtcatcgcgatccacaatgccatgaaattgcggtgagt
acggcattgatgagtgccagcaacccacttgcattttgatctattaaattgaacagctcaattccatcatcgctcatcaca
cgtgaagtggtatgggagtttggaccccgttcatctaaggcaacaaagtcatgatagctatcttccgtcctggtttcatc
cctccaaaatcctcgtggatacggcaaattgtcgatgacttgctaacggactaaagtacagggtacgataaccatccgatc
gaatgggcgaaagggacataccaaatataccctcctcgccccattggaagttaataccagcgacattgaaatcgccctc
aatggatcggggaaaaaatacgaatgtgccgagaatctccaggacttgttcagttcccaggagtaagcaatcaagccgta
tcccaattcatcggcaaaataggcatatgcatcatcgcaatttttgcctatatccacggcaatgttagctatgaaagtat
ttggatttgtgtccacgccaggtagctcgtatctccgaattcgcgtatccgtggtcaagtcaaagacatttaccgcatag
gggcacggattagtGGTGGTATTGCCGATGCCCACGGtcgacCGAGCTCGCCCGGGGATC
TAATTCAATTAGAGACTAAT
TCAATTAGAGCTAATTCAATTAGGATCCAAGCTTATCGATTTCGAACCCTCGA
CCGCCGGAGTATAAATAGAGGCGCTTC
GTCTACGGAGCGACAATTCAATTCAAACAAGCAAAGTGAACACGTCGCTAAG
CGAAAGCTAAGCAAATAAACAAGCGCAG
CTGAACAAGCTAAACAATCGGCTCGAAGCCGGTCGCCACCatgGCCTCCTCCGA
GGACGTCATCAAGGAGTTCATGCGCT
TCAAGGTGCGCATGGAGGGCTCCGTGAACGGCCACGAGTTCGGAGATAGGG
CGAGGGCGAGGGCCGCCCCTACGAGGGC
ACCCAGACCGCCAACGTGAAGGTGACCAAGGGCGGCCCCCTGCCCTTCGCCTG
GGACATCCTGTCCCCCCAGTTCCAGTA
CGGCTCCAAGGTGTACGTGAAGCACCCCGCCGACATCCCCGACTACAAGAAG
CTGTCCTTCCCCGAGGGCTTCAAGTGGG
AGCGCGTGATGAACTTCGAGGACGGCGGGTGGTGACCGTGACCCAGGACTC
CTCCCTcCAGGACGGCTCCTTCATCTAC
AAGGTGAAGTTCATCGGCGTGAACTTCCCCTCCGACGGCCCCGTAATGCAGAA
GAAGACTATGGGCTGGGAGGCgTCCAC
CGAGCGCCTGTACCCCCGCGACGGCGTGCTGAAGGGCGAGATCCACAAGGCC
CTGAAGCTGAAGGACCGGCGGCCACTACC
TGGTGGAGTTCAAGTCCATCTACATGGCCAAGAAGCCCGTGCAGCTGCCCGGC
TACTACTACGTGGACTCCAAGCTGGAC
ATCACCTCCCACAACGAGGACTACACCATCGTGGAGCAGTACGAGCGCGCCG
AGGGCCGCCACCACCTGTTCCTGTAGgg
ggcGCGACTCTAGATCATAATCAGCCATACCACATTTGTAGAGGTTTTACTTGCT
TTAAAAAACCTCCCACACCTCCCCC
TGAACCTGAAACATAAAATGAATGCAATTGTTGTTGTTAACTTGTTTATTGCA
GCTTATAATGGTTACaaataaaGCAAT
AGCATCACAAATTTCACaaataaaGCATTTTTTTCACTGCATTCTAGTTGTGGTTTG
TCCAAACTCATCAATGTATCtta
AAGCTTATCGATACGCGTACgctagcACAAAAGCTGGAGCTCCTGCAGGTTGTTG
GTTGGCACACCACAAATATACTGTT
GCCGAGCACAATTGTCTAGAATGCATACGCATTAAGCGAACATTAAAAAGAT
GTGAAAACATAACTATTATGTCTAAATA
AACACACGTCAGATGTATGTACGTCAACGGAAAACCATTGTCTATATATTACA
ATTACTAAATACATACCAAATTGAATA
CATATTGATGAAAAATAATAAATACTGGCGAAAGCAAAAAAACGAAACATTT
TTATTTTATTGAACAACTCTCAGGCTCC
AGGTAGGCAAAAAAGCACCGACTCGGTGCCACTTTTTCAAGTTGATAACGGAC
TAGCCTTATTTTAACTTGCTATTTCTA
GCTCTAAAACCGGTTCCAGTGTCCAAAACCGACGTTAAATTGAAAATAGGTCT
ATATATACGAACTGAGTCTGGAAAAAG
AAGTTGAGAATTATAAAAAGTAGTGAGCACTGGCGCCCTCTCTGCTTGGCGAG
CTAACCTTTTCGCCTCTTGGCTGAGTA
GGTGGCGTTTCATTCTACTCTGTAAAATTAATGTAGAATTGAAACACTATGGT
CAAAAAATACTTAGGGGCATAAGTTAT
AAAACGTATGAAATGAATTTTTATCAACCTGGGCTATTCAAAAATTTTCGAAT
TATTTTATGTATTTTTTTTAATCGTTT
TTCATTATAGGTTAAAATACACTTTAAAAGGAATTCTTTCCTGTAAAATAAAT
ATAAATAAATATGCTTTATTGACAGAA
AATTTGATGTTTTTGTATTTGAGTAGGAGCAATCACAGGTGAGCAAAAAAGCA
CCGACTCGGTGCCACTTTTTCAAGTTG
ATAACGGACTAGCCTTATTTTAACTTGCTATTTCTAGCTCTAAAACtgggagcagtag
cacacatgcCGAAGTTCACCCG
GATATCTTTCCTATATATACTGTACTCTTGCAGAATATGTACTTGGGAATCATA
AGGATGCGAAGGACGTACGCGCTGTG
TCTGTAACTAAGGGGTTGCTTTCGGGTACCTACCATGCCAATGATGGGACTAC
CGCCGGATAGGAATCCCTTTTGGGTGC
ACACCGTCACCTCCAGTTCCGTCTGCCTGAGTTCGGCAATGGAAATCAGGTAC
TCAAAGGATGCATCGTAGACGGGGTTG
CAGTTGTCCTTGATCACGCTCGTCTTGCGTTTGCACTCCTTGGTGCGTCCAGGC
AACAGATACAGCTTAACATACGGATC
GGGGATATTGCTGGGATCGCGAAGTGGTATCTTCTGGATTTTGTGTATGGTCA
CGTCTAGTTTTTGACGCTGGGCGCTGT
AGCGGATCGACAACTGCATTCGTCCAAGGCCATGTTCGCCFFFTGAGGAGGT
GGAGTCTGGAAAGCGGTGGGTCAGCACA
GAGGTGGCCAGCTCGCTGATGGGTTTTTCGCTGGAGGTGGAGGCCGACATGGC
CGGGGAGATTTTCGTAGCCGCCACAGG
ATCTTCTTCACTGATGGGCGATTCGGGCGGACAAACGCTTGCTGGATGCCTGAG
ATTCCTAAAAGCAATGGCAAATTAGAT
TCAATTAAAACAAAAATTGTTTATATAACAAAGTAAAAGAAAAAAAAAAAGA
ATCACAGATTCTCCCCGACGGGGAATTG
AACCCCGGTCTCCCGCGTGACAGGCGGGGATACTAACCACTATACTATCGAGG
ACGTTGAAAATgacgtcttccagtgtc
caaaacccacagccgaccacactcatccactttaatgcggtaggcagtggtaatactgttggcgcaatctccgctgtat
ttgagcgccaatctggatacggaattagctccggtgaacccgtcaaactgcggtccatgtttatataggtcagagtggcc
ggaatccctaaaaaataaaataggagaattagcagggccaaactattattgaggcactaaataaagtttgttttgaatat
ttaattaactggaatttctgcctaaattccttctttggattattatactaagcaatgttgcaagttatcgctgtctgttt
tctggataatagtaaatgcatacagattgtaaaggtaaattagaatcgcttatgtttggcatttaattatttatacct
atttgtcattattttggacacaaggaatcataagcaatggtaactgccgtgctaatggctcttttttctgcttgtacatc
tactggaagtttatgttaaaagatccccggctatatgtacttcagtcataattggcactattgcaagttgcgtgcaattt
gcgatgtcattagccgtctgaagcaattgcgtctgacatcattatcaatatgtataccatatatcgctgttaaatgtata
tgtggccattggcagtatttcgaccgatttccgctccagttcttttccttctttatttcttcgttaattgaatttaaaga
catttattgtttctgggaacttttaatgtttttatagcaggcgagtgagactgcaacgaccagaaaatatagcttttatc
gatttaccgacccatcttcttaaaaatcaaaccaggcgtttttgctttacaaagtaaccgggggttgcgataaatataca
tttgagcataatgcacctttcaatctttatttaattcataacttttaaaataacaactaattcagtcaaatataaagta
tttgaacaaatttatattttgacatgtgctctttcagtcctaaaacctcgcaacAAGGGCGAATTCTGCAGATATC
CATC
ACACTGGCGGCCGCTCGAGCATGCATCTAGAGGGCCCAATTCGCCCTATAGTG
AGTCGTATTACAATTCACTGGCCGTCG
TTTTACAACGTCGTGACTGGGAAAAACCCTGGCGTTACCCAACTTAATCGCCTT
GCAGCACATCCCCCTTTCGCCAGCTGG
CGTAATAGCGAAGAGGCCCGCACCGATCGCCCTTCCCAACAGTTGCGCAGCCT
GAATGGCGAATGGACGCGCCCTGTAGC
GGCGCATTAAGCGCGGCGGGTGTGGTGGTTAGCCGCAGCGTGACCGCTACACT
TGCCAGCGCCCTAGCGCCCGCTCCTTT
GCGTTTCTTCCCTTCCTTTCTCGCCACGTTCGCCGGCTTTCCCCGTCAAGCTCTA
AATCGGGGGCTCCCTTTAGGGTTCC
GATTTAGTGCTTTACGGCACCTCGACCCCAAAAAACTTGATTAGGGTGATGGT
TCACGTAGTGGGCCATCGCCCTGATAG
ACGGTTTTTCGCCCTTTGACGTTGGAGTCCACGTTCTTTAATAGTGGACTCTTG
TTCCAAACTGGAACAACACTCAACCC
TATCTCGGTCTATTCTTTTGATTTATAAGGGATTTTGCCGATTTCGGCCTATTG
GTTAAAAAATGAGCTGATTTAACAAA
AATTTAACGCGAATTTTAACAAAATTCAGGGCGCAAGGGCTGCTAAAGGAAG
CGGAACACGTAGAAAGCCAGTCCGCAGA
AACGGTGCTGACCCCGGATGAATGTCAGCTACTGGGCTATCTGGACAAGGGA
AAACGCAAGCGCAAAGAGAAAGCAGGTA
GCTTGCAGTGGGCTTACATGGCGATAGCTAGACTGGGCGGTTTTATGGACAGC
AAGCGAACCGGAATTGCCAGCTGGGGC
GCCCTCTGGTAAGGTTGGGAAGCCCTGCAAAGTAAACTGGATGGCTTTCTTGC
CGCCAAGGATCTGATGGCGCAGGGGAT
CAAGATCTGATCAAGAGACAGGATGAGGATCGTTTCGCATGATTGAACAAGA
TGGATTGCACGCAGGTTCTCCGGCCGCT
TGGGTGGAGAGGCTATTCGGCTATGACTGGGCACAACAGACAATCGGCTGCTC
TGATGCCGCCGTGTTCCGGCTGTCAGC
GCAGGGGCGCCCGGTTCTTTTTGTCAAGACCGACCTGTCCGGTGCCCTGAATG
AACTGCAGGACGAGGCAGCGCGGCTAT
CGTGGCTGGCCACGACGGGCGTTCCTTGCGCAGCTGTGCTCGACGTTGTCACT
GAAGCGGGAAGGGACTGGCTGCTATTG
GGCGAAGTGCCGGGGCAGGATCTCCTGTCATCCCACCTTGCTCCTGCCGAGAA
AGTATCCATCATGGCTGATGCAATGCG
GCGGCTGCATACGCTTGATCCGGCTACCTGCCCATTCGACCACCAAGCGAAC
ATCGCATCGAGCGAGCACGTACTCGGA
TGGAAGCCGGTCTTGTCGATCAGGATGATCTGGACGAAGAGCATCAGGGGCT
CGCGCCAGCCGAACTGTTCGCCAGGCTC
AAGGCGCGCATGCCCGACGGCGAGGATCTCGTCGTGACCCATGGCGATGCCT
GCTTGCCGAATATCATGGTGGAAAATGG
CCGCTTTTCTGGATTCATCGACTGTGGCCGGCTGGGTGTGGCGGACCGCTATC
AGGACATAGCGTTGGCTACCCGTGATA
TTGCTGAAGAGCTTGGCGGCGAATGGGCTGACCGCTTCCTCGTGCTTTACGGT
ATCGCCGCTCCCGATTCGCAGCGCATC
GCCTTCTATCGCCTTCTTGACGAGTTCTTCTGAATTGAAAAAGGAAGAGTATG
AGTATTCAACATTTCCGTGTCGCCCTT
ATTCCCTTTTTTGCGGCATTTTGCCTTCCTGTTTTTGCTCACCCAGAAACGCTGG
TGAAAGTAAAAGATGCTGAAGATCA
GTTGGGTGCACGAGTGGGTTACATCGAACTGGATCTCAACAGCGGTAAGATCC
TTGAGAGTTTTCGCCCCGAAGAACGTT
TTCCAATGATGAGCACTTTTAAAGTTCTGCTATGTGGCGCGGTATTATCCCGTA
TTGACGCCGGGCAAGAGCAACTCGGT
CGCCGCATACACTATTCTCAGAATGACTTGGTTGAGTACTCACCAGTCACAGA
AAAGCATCTTACGGATGGCATGACAGT
AAGAGAATTATGCAGTGCTGCCATAACCATGAGTGATAACACTGCGGCCAACGT
TACTTCTGACAACGATCGGAGGAACCGA
AGGAGCTAACCGCTTTTTTGCACAACATGGGGATCATGTAACTCGCCTTGAT
CGTTGGGAACCGGAGCTGAATGAAGCC
ATACCAAACGACGAGCGTGACACCACGATGCCTGTAGCAATGGCAACAACGT
TGCGCCAAACTATTAACTGGCGAACTACT
TACTCTAGCTTCCCGGCAACAATTAATAGACTGGATGGAGGCGGATAAAGTTG
CAGGACCACTTCTGCGCTCGGCCCTTC
CGGCTGGCTGGTTTATTGCTGATAAATCTGGAGCCGGTGAGCGTGGGTCTCGC
GGTATCATTGCAGCACTGGGGCCAGAT
GGTAAGCCCTCCCGTATCGTAGTTATCTACACGACGGGGAGTCAGGCAACTAT
GGATGAACGAAATAGACAGATCGCTGA
GATAGGTGCCTCACTGATTAAGCATTGGTAACTGTCAGACCAAGTTTACTCAT
ATATAACTTTAGATTGATTTAAAACTTC
ATTTTTAATTTAAAAGGATCTAGGTGAAGATCCTTTTTGATAATCTCATGACCA
AAATCCCTTAACGTGAGTTTTCGTTC
CACTGAGCGTCAGACCCCGTAGAAAAGATCAAAGGATCTTCTTGAGATCCTTT
TTTTCTGCGCGTAATCTGCTGCTTGCA
AACAAAAAAACCACCGCTACCAGCGGTGGTTTGTTTGCCGGATCAAGAGCTAC
CAACTCTTTTTCCGAAGGTAACTGGCT
TCAGCAGAGCGCAGATACCAAATACTGTTCTTCTAGTGTAGCCGTAGTTAGGC
CACCATTCAAGAACTCTGTAGCACCG
CCTACATACCCGCTCTGCTAATCCTGTTACCAGTGGCTGCTGCCAGTGGCGAT
AAGTCGTGTCTTACCGGGTTGGACTC
AAGACGATAGTTACCGGATAAGGCGCAGCGGTCGGGCTGAACGGGGGGTTCG
TGCACACGCCCAGCTTGGAGCGAACGA
CCTACACCGAACTGAGATACCTACAGCGTGAGCTATGAGAAAGCGCCACGCTT
CCCGAAGGGAGAAAGGCGGACAGGTAT
CCGGTAAGCGGCAGGGTCGGAACAGGAGAGCGCACGAGGGAGCTTCCAGGG
GGAAACGCCTGGTATCTTTATAGTCCTGT
CGGGTTTCGCCACCTCTGACTTGAGCGTCGATTTTTGTGATGCTCGTCAGGGGG
GCGGAGCCTATGGAAAAACGCCAGCA
ACGCGGCCTTTTTACGGTTCCTGGCCTTTTGCTGGCCTTTTGCTCACATGTTCTT
TCCTGCGTTATCCCCTGATTCTGTG
GATAACCGTATTACCGCCTTTGAGTGAGCTGATACCGCTCGCCGCAGCCGAAC
GACCGAGCGCAGCGAGTCAGTGAGCGA
GGAAGCGGAAGAGCGCCCAATACGCAA

TABLE 2
Phenotypic and molecular analysis of 104 isogenic F2 lines.

| | | Donor | N+/ | y+ | NAx | | |
|---|---|---|---|---|---|---|---|
| | | or | Ax16 | or | region | | |
| line # | DsRed | Receiver | phenotype | y− | Sequence | Category | Comments |
| A1 | + | D | Ax | y− | | DON | |
| A2 | + | D | Ax | y− | | DON | |
| A4 | + | R | Ax | y− | Ax16 | DCR | |
| A5 | + | R | Ax | y− | Ax16 | DCR | |
| A6 | + | R | Ax | y− | Ax16 | DCR | |
| A7 | + | D | Ax | y− | | DON | |
| A8 | + | D | Ax | y− | | DON | |
| A9 | + | D | Ax | y− | | DON | |
| A10 | + | D | Ax | y− | | DON | |
| A11 | − | R | Ax | y− | 3 nt deletion | ACR | new Ax |
| | | | | | at cut site | | allele |
| A12 | + | R | Ax | y− | Ax16 | DCR | |
| A13 | + | R | N+ | y− | N+ | RCR | |
| A14 | + | D | Ax | y− | | DON | |
| A15 | + | D | Ax | y− | | DON | |
| C25 | + | R | Ax | y− | Ax16 | DCR | |
| C26 | + | R | Ax | y− | Ax16 | DCR | |
| C27 | + | R | Ax | y− | Ax16 | DCR | |
| C28 | − | R | N+ | y− | N+ | INR | |
| C29 | + | D | Ax | y− | | DON | |
| D31 | − | R | Ax | y− | Ax16 | ACR | |
| D33 | + | R | Ax | y− | Ax16 | DCR | |
| D34 | + | D | Ax | y− | | DON | |
| D35 | + | D | Ax | y− | | DON | |
| D36 | − | R | N+ | y− | N+ | INR | |
| D37 | − | R | N+ | y− | N+ | INR | |
| D39 | + | D | Ax | y− | | DON | |
| E41 | + | D | Ax | y− | | DON | |
| E42 | + | R | Ax | y− | Ax16 | DCR | |
| E43 | + | D | Ax | y− | | DON | |
| E44 | − | D | Ax | y− | | DON | recombination |
| E45 | − | R | Ax | y− | Ax16 | ACR | |
| E47 | + | R | Ax | y− | Ax16 | DCR | |
| E48 | − | R | Ax | y− | Ax16 | ACR | |
| E49 | + | R | Ax | y− | Ax16 | DCR | |
| F50 | + | D | Ax | y− | | DON | |
| F51 | − | R | Ax | y− | Ax16 | ACR | |
| G52 | − | R | N+ | y− | C to A sub, | INR | cut-resistant |
| | | | | | at position | | N+ allele |
| | | | | | −1 | | |
| G53 | − | R | N+ | y− | N+ | INR | |
| G54 | − | R | Ax | y− | Ax16 | ACR | |
| H55 | − | R | N+ | y− | N+ | INR | |
| H56 | + | D | Ax | y− | | DON | |
| H57 | + | D | Ax | y− | | DON | |
| H58 | + | D | Ax | y− | | DON | |
| H59 | − | R | Ax | y− | Ax16 | ACR | |
| I60 | − | R | Ax | y− | Ax16 | ACR | |
| I61 | + | D | Ax | − | | DON | |
| I62 | − | R | N+ | y− | N+ | INR | |
| I63 | + | R | Ax | y− | Ax16 | DCR | |
| I64 | + | D | Ax | y− | | DON | |
| I65 | + | D | Ax | y− | | DON | |
| I66 | + | D | Ax | y− | | DON | |
| I67 | + | D | Ax | y− | | DON | |
| I68 | + | D | Ax | y− | | DON | |
| K69 | + | D | Ax | y− | | DON | |
| K70 | − | R | N+ | y− | N+ | INR | |
| K71 | + | D | Ax | y− | | DON | |
| K72 | + | D | Ax | y− | | DON | |
| K73 | + | D | Ax | y− | | DON | |
| K74 | + | D | Ax | y− | | DON | |
| K76 | + | R | Ax | y− | Ax16 | DCR | |
| K77 | − | R | Ax | y− | Ax16 | ACR | |
| K78 | + | D | Ax | y− | | DON | |
| K79 | − | R | Ax | y− | Ax16 | ACR | |
| K80 | − | D | Ax | y− | | DON | recombination |
| K81 | + | D | Ax | y− | | DON | |
| K82 | − | R | Ax | y− | Ax16 | ACR | |
| K83 | − | R | Ax | y− | Ax16 | ACR | |
| K84 | + | D | Ax | y− | | DON | |
| K85 | + | D | Ax | y− | | DON | |
| K87 | + | D | Ax | y− | | DON | |
| K88 | + | R | Ax | y− | Ax16 | DCR | |
| K89 | + | D | Ax | y− | | DON | |
| K90 | + | D | Ax | y− | | DON | |
| K91 | + | D | Ax | y− | | DON | |
| L92 | + | D | Ax | y− | | DON | |
| L93 | + | R | Ax | y− | Ax16 | DCR | |
| L94 | + | R | Ax | y− | Ax16 | DCR | |
| L95 | + | D | Ax | y− | | DON | |
| L96 | + | D | Ax | y− | | DON | |
| L97 | + | D | Ax | y− | | DON | |
| L98 | + | D | Ax | y− | | DON | |
| L99 | + | R | Ax | y− | Ax16 | DCR | |
| M100 | − | R | Ax | y− | Ax16 | ACR | |
| M101 | − | R | N+ | y− | N+ | IR | |
| M102 | + | R | Ax | y− | Ax16 | DCR | |
| M103 | + | R | Ax | y− | 3 nt deletion | DCR | new Ax |
| | | | | | at cut site | | allele |
| M104 | − | R | N+ | y− | N+ | IR | |
| M105 | + | D | Ax | y− | | DON | |
| M106 | + | D | Ax | y− | | DON | |
| M107 | + | D | Ax | y− | | DON | |
| M108 | + | D | Ax | y− | | DON | |
| M109 | − | R | Ax | y− | Ax16 | ACR | |
| M110 | + | R | Ax | y− | Ax16 | DCR | |
| M111 | + | R | Ax | y− | Ax16 | DCR | |
| M112 | + | R | Ax | y− | Ax16 | DCR | |
| M113 | + | D | Ax | y− | | DON | |
| M114 | + | D | Ax | y− | | DON | |
| M115 | − | R | N+ | y− | N+ | INR | |
| M117 | + | D | Ax | y− | | DON | |
| M118 | + | D | Ax | y− | | DON | |
| M119 | − | R | N+ | y− | N+ | INR | |
| M120 | + | R | Ax | y− | Ax16 | DCR | |
| M121 | + | D | Ax | y− | | DON | |
| M122 | + | D | Ax | y− | | DON | |

Table 2: Phenotypic, and molecular analysis of 104 isogenic F2 female lines. Column 1: line number. Column 2: Presence (+) or absence (−) of the DsRed-marked ccN CopyCat element. Column 3: origin of the founder chromosome (Donor: D, or Receiver: R), as determined by sequencing a polymorphic site located 6.5 Kb upstream of the Ax16 mutation (FIG. 8A). Receiver lines are highlighted in green. Column 4: Notch wing phenotype, Abruptex (Ax) or wild-type (N+). Column 5: Yellow allele (only y- alleles were recovered). Column 6: Sequence analysis of Ax region from Receiver chromosomes, revealing a strict correspondence between the phenotypic and molecular analysis. In addition, two Ax alleles (lines 11 and 103) were created by non-homologous end joining rather than allelic conversion. Column 7: Chromosome category. DON: Donor. DCR: Double converted receiver. INR: Intact receiver. ACR: Ax only converted receiver. RCR: DsRed only converted receiver. Column 8: Comments. For lines 44 and 80, recombination separated Ax form the DsRed ccN CopyCat element. For lines 11 and 103, a new Ax alleles was created.

TABLE 3
Summary of phenotypic analysis of 104 isogenic lines

| Categories | Abbreviation | #Lines | Percentage |
|---|---|---|---|
| Donor | DON | 54 | 51.92% |
| Double Conversion | DCR | 23 | 22.11% |
| Intact receiver | INR | 12 | 11.53% |
| Ax only Conversion | ACR | 14 | 13.46% |
| DsRed only Conversion | RCR | 1 | 0.96% |
| Total | | 104 | |
| Categories | Includes | #Lines | Percentage |
| DsRed- | ACR + INR | 28 | 27% |
| DsRed+ | DON + DCR + RCR | 76 | 75% |
| N+ | INR + RCR | 13 | 12.50% |
| NAx | DON + ACR + DCR | 91 | 87.50% |
| DsRed Conversion | DCR + RCR | 24 | 48% |
| NAx Conversion | DCR + ACR | 37 | 74% |

Table 3: Summary of the different categories of molecular events determined on receiver versus donor chromosomes for the 104 isogenic lines and accompanying abbreviation key Table 2.

The embodiments of the invention disclosed herein are exemplary, and other embodiments and variations of the invention will be apparent to those skilled in the art and are intended to be encompassed herein.

### REFERENCES

1 DiCarlo, J. E., Chavez, A., Dietz, S. L., Esvelt, K. M. & Church, G. M. RNA-guided gene drives can efficiently bias inheritance in wild yeast, bioRxiv, doi:http://dx.doi.org/10.1101/013896 (2015).
2 Gantz, V. M. & Bier, E. The mutagenic chain reaction: a method for converting heterozygous to homozygous mutations. Science 348, 442-444, doi:science.aaa5945 [pii]10.1126/science.aaa5945 (2015).
3 Gantz, V. M. et al. Highly efficient Cas9-mediated gene drive for population modification of the malaria vector mosquito Anopheles stephensi, Proc Natl Acad Sci USA 112, E6736-6743, doi:1521077112 [pii] 10.1073/pnas.1521077112 (2015).
4 Grunwald, H. A. et al. Super-Mendelian inheritance mediated by CRISPR-Cas9 in the female mouse germline. Nature In Press (2019).
5 Hammond, A. et al. A CRISPR-Cas9 gene drive system targeting female reproduction in the malaria mosquito vector Anopheles gambiae, Nat Biotechnol 34, 78-83, doi:10.1038/nbt.3439 (2016).
6 Gantz, V. M. & Bier, E. The dawn of active genetics. Bioessays 38, 50-63, doi:10.1002/bies.201500102 (2016).
7 Xu, X. S., Gantz, V. M., Siomava, N. & Bier, E. CRISPR-Cas9 and active genetics-based trans-species replacement of the endogenous Drosophila kni-L2 CRM reveals unexpected complexity. Elife 6, doi:10.7554/eLife.30281 (2017).
8 Housden, B. E. et al. Loss-of-function genetic tools for animal models: cross-species and cross-platform differences. Nat Rev Genet 18, 24-40, doi:10.1038/nrg.2016.118 (2017).
9 Komor, A. C., Badran, A. H. & Liu, D. R. CRISPR-Based Technologies for the Manipulation of Eukaryotic Genomes. Cell 168, 20-36, doi:10.1016/j.cell.2016.10.044 (2017).
10 Paquet, D. et al. Efficient introduction of specific homozygous and heterozygous mutations using CRISPR-Cas9. Nature 533, 125-129, doi:10.1038/nature17664 (2016).
11 Kelley, M. R., Kidd, S., Deutsch, W. A. & Young, M. W. Mutations altering the structure of epidermal growth factor-like coding sequences at the Drosophila Notch locus. Cell 51, 539-548 (1987).
12 Lin, C. C. & Potter, C. J. Non-Mendelian Dominant Maternal Effects Caused by CRISPR-Cas9 Transgenic Components in Drosophila melanogaster. G3 (Bethesda), doi:10.1534/g3.116.034884 (2016).
13 Champer, J. et al. Novel CRISPR-Cas9 gene drive constructs reveal insights into mechanisms of resistance allele formation and drive efficiency in genetically diverse populations. PLoS Genet 13, e1006796, doi:10.1371/journal.pgen.1006796 (2017).
14 Lehmann, R., Dietrich, U., Jimenez, F. & Campos-Ortega, J. A. Mutations of early neurogenesis in Drosophila. Wilehm Roux Arch Dev Biol 190, 226-229, doi:10.1007/BF00848307 (1981).
15 Mehta, A. & Haber, J. E. Sources of DNA double-strand breaks and models of recombinational DNA repair. Cold Spring Harb Perspect Biol 6, a016428, doi:10.1101/cshperspect.a016428 (2014).
16 Joyce, E. F., Paul, A., Chen. K. E., Tanneti, N. & McKim, K. S. Multiple barriers to nonhomologous DNA end joining during meiosis in Drosophila. Genetics 191, 739-746, doi:10.1534/genetics.112.140996 (2012).
17 Wei, D. S. & Rong, Y. S. A genetic screen for DNA double-strand break repair mutations in Drosophila. Genetics 177, 63-77, doi:10.1534/genetics.107.077693 (2007).
18 Do, A. T., Brooks, J. T., Le Neveu, M. K. & LaRocque, J. R. Double-strand break repair assays determine pathway choice and structure of gene conversion events in Drosophila melanogaster. G3 (Bethesda) 4, 425-432, doi:10.1534/g3.113.010074 (2014).
19 Ewen-Campen, B. & Perrimon, N. ovo(D) Co-selection: A Method for Enriching CRISPR-Cas9-Edited Alleles in Drosophila. G3 (Bethesda) 8, 2749-2756, doi:10.1534/g3.118.200498 (2018).
20 Roth, T. L. et al. Reprogramming human T cell function and specificity with non-viral genome targeting. Nature 559, 405-409, doi:10.1038/s41586-018-0326-5 (2018).
21 Hu, J. H. et al. Evolved Cas9 variants with broad PAM compatibility and high DNA specificity. Nature 556, 57-63, doi:10.1038/nature26155 (2018).
22 Nishimasu, H. et al. Engineered CRISPR-Cas9 nuclease with expanded targeting space. Science 361, 1259-1262, doi:10.1126/science.aas9129 (2018).
23 Xiong, H. et al. Natural Variation in OsLG3 Increases Drought Tolerance in Rice by Inducing ROS Scavenging. Plant Physiol 178, 451-467, doi:10.1104/pp.17.01492 (2018).
24 Lemmon, Z. H. et al. Rapid improvement of domestication traits in an orphan crop by genome editing. Nat Plants 4, 766-770, doi:10.1038/s41477-018-0259-x (2018).
25 Dixon, L. E. et al. TEOSINTE BRANCHED1 Regulates Inflorescence Architecture and Development in Bread Wheat (Triticum aestivum), Plant Cell 30, 563-581, doi:10.1105/tpc.17.00961 (2018).
26 Wissuwa, M., Kretzschmar, T. & Rose, T. J. From promise to application: root traits for enhanced nutrient capture in rice breeding. J Exp Bol 67, 3605-3615, doi:10.1093/jxb/erw061 (2016).
27 Ffrench-Constant, R. H., Williamson, M. S., Davies, T. G. & Bass, C. Ion channels as insecticide targets. J Neurogenet 30, 163-177, doi:10.1080/01677063.2016.1229781 (2016).
28 Brito, L. P. et al. Assessing the effects of Aedes aegypti kdr imitations on pyrethroid resistance and its fitness cost. PLoS One 8, e60878, doi:10.1371/journal.pone.0060878 (2013).
29 Hanai, D., Hardstone Yoshimizu, M. & Scott, J. G. The insecticide Resistance Allele kdr-his has a Fitness Cost in the Absence of Insecticide Exposure. J Econ Entomol 111, 2992-2995, doi:10.1093/jee/toy300 (2018).
30 Platt, R. J. et al. CRISPR-Cas9 Knockin Mice for Genome Editing and Cancer Modeling. Cell 159, 440-455, doi:S0092-8674(14)01163-5 [pii] 10.1016/j.cell.2014.09.014 (2014).
31 Churcher, T. S., Lissenden, N., Griffin, J. T., Worrall, E. & Ranson, H. The impact of pyrethroid resistance on the efficacy and effectiveness of bednets for malaria control in Africa. Elife 5, doi:10.7554/eLife.16090 (2016).
32 Glunt, K. D. et al. Empirical and theoretical investigation into the potential impacts of insecticide resistance on the effectiveness of insecticide-treated bed nets. Evol Appl 11, 431-441, doi:10.1111/eva.12574 (2018).
33 Gloor, G. B. et al. Type I repressors of P element mobility. Genetics 135, 81-95 (1993).
34 Akbari, O. S. et al. Safeguarding gene drive experiments in the laboratory. Science 349, 927-929, doi:science.aac7932 [pii] 10.1126/science.aac7932 (2015).
35 Org., C. G. D. R. i. N.-H., Conduct, R. f. R., Sciences, B. o. L., Studies, D. E. a. L. & Med., N. A. S. E. Gene Drives on the Horizon: Advancing Science, Navigating Uncertainty, and Aligning Research with Public Values. (THE NATIONAL ACADEMIES PRESS, Washington D.C., 2016).
36 Sturtevant, A. H., J. Animal Behavior 5, 351-366, 1915.

1. A method of introducing a preferred nucleotide sequence into a genome by allelic-drive copy-cutting, comprising genomically replacing a non-preferred nucleotide sequence with that of a preferred allele of the same gene using an allelic-drive element that comprises first and second guide RNAs and a cleavage resistant preferred allele, wherein a Cas endonuclease guided by the first guide RNA copies the allelic-drive element, and a Cas endonuclease guided by the second guide RNA cuts the non-preferred allele, but not the preferred allele, and copies the cleavage resistant preferred allele into a double stranded DNA break created by the second guide RNA by homology-directed repair (HDR)-mediated repair.

2. A method of introducing a preferred nucleotide sequence into a genome by allelic-drive copy-grafting, comprising genomically replacing a non-preferred nucleotide sequence with that of a preferred allele of the same gene using an allelic-drive element that comprises a first and a second guide RNAs and a cleavage resistant site adjacent to the preferred allele, wherein Cas endonuclease guided by the first guide RNA copies the allelic-drive element, and the second guide RNA cuts the non-preferred allele, but not the preferred allele, that is associated with the cleavage resistant sequence residing within less than 100 nucleotides of the preferred allele, and copies the cleavage resistant sequence together with the preferred adjacent allele by virtue of a short range single stranded resection step which occurs during homology-directed repair (HDR)-mediated repair.

3. The method of claim 1, wherein the Cas endonuclease is not integrated into the allelic-drive element.

4. The method of claim 1, wherein Cas endonuclease is integrated into the allelic drive element and transmission of both alleles is super-Mendelian.

5. The method of claim 1, wherein allelic conversion of both alleles in a second filial generation is at least 40%, 50%, 60% or 70%

6. The method of claim 2, wherein allelic conversion of both alleles in a second filial generation is at least 80%, 85%, 90%, 95% or 97%.

7. The method of claim 2, wherein the second guide RNA targets the second chromosome at a sensitive portion within less than 80, 60, 40, 25, 20, 10 or 5 nucleotides of the non-preferred allele.

8. The method of claim 1, wherein progeny, which maintain an association between a first allele comprising an allelic-drive element and a second uncleavable allele, survive in the presence of Cas9.

9. The method of claim 1, wherein perduring Cas9-gRNA complexes are transmitted maternally for one generation in the absence of Cas9 or gRNA transgenes.

10. The method of claim 1, wherein the allelic-drive element is inserted into an essential gene required for viability or fertility and also carries functional recoded sequences of the essential gene rendering the allelic drive element viable in a homozygous or hemizygous state.

11. The method of claim 10, wherein the functional recoded sequences of the essential gene are recoded as a direct in-frame fusion with recoded cDNA sequences inserted at the 5 end of the allelic drive element abutting the guide RNA cut site.

12. The method of claim 10, wherein the essential gene is Notch, Rab11, Rab5, Rab1; Prosalpha1, Dpp=decapentaplegic, EGF-Receptor; Mre11, Spo11, cinnabar=kynurenine hydroxylase; doublesex; a gene encoding a male-specific tubulin subunit; cardinal, ENa=sodium ion channel, or FREP1.

13. A method of selectively eliminating an allele of a gene generated by non-homologous end joining (NHEJ) comprising utilizing a lethal mosaicism technique, wherein NHEJ-induced drive-resistant, non-functional, or loss-of-function alleles of an essential gene are dominantly eliminated in progeny due to maternal perdurance of Cas/guide RNA complexes targeting the paternal allele.

14. A method of germline editing comprising repair of a cleavage-sensitive allele with sequences provided by a cut-resistant allele present on the homologous chromosome in heterozygous individuals.

15. The method of claim 1, wherein the alleles are assembled in plants to provide drought resistance, higher crop yields, optimal architectures, or rapid growth.

16. The method of claim 1, wherein the alleles are assembled in insects to reverse pesticide resistance in pest species or to favor genetic variants that prevent host species from serving as disease vectors.

17. An organism made by a method of claim 1.
